(19) United States
(12) Patent Application Publication
(10) Pub. No.: US 2026/0097052 A1
(43) Pub. Date: Apr. 9, 2026

(54) NOVEL ORAL CANNABINOID FORMULATIONS

(71) Applicant: Esolate Pharmaceuticals Ltd, London (GB)

(72) Inventors: Hardin Matthew Jackson, Burlington (CA); Devin Douglas Machin, Toronto (CA); Jinhee Oh, Hamilton (CA); Venkat Reddy Goskonda, Phoenix, AZ (US); Andrew Jonathan Saich, Hungerford (GB)

(21) Appl. No.: 19/418,328

(22) Filed: Dec. 12, 2025

Related U.S. Application Data
(63) Continuation of application No. PCT/EP2024/081400, filed on Nov. 6, 2024.

(30) Foreign Application Priority Data
Nov. 6, 2023 (GB) 2316995.6
Jan. 8, 2024 (GB) 2400238.8
Jul. 11, 2024 (GB) 2410075.2

(51) Int. Cl. A61K 31/00; A61K 9/48

(52) U.S. Cl. CPC A61K 31/658; A61K 9/4858; A61K 9/4866

(57) ABSTRACT

The present disclosure relates to novel pharmaceutical formulations, including solid oral dosage forms comprising the non-psychoactive cannabinoid cannabidiol (CBD). The disclosure further relates to methods of manufacture of the pharmaceutical formulations and the use of the formulations to treat diseases and conditions.

### CROSS-REFERENCE TO RELATED APPLICATIONS

This application is a continuation of International Application No. PCT/EP2024/081400, filed on Nov. 6, 2024, which claims the benefit of priority to GB Patent Application No. 2410075.2, filed on Jul. 11, 2024, GB Patent Application No. 2400238.8, filed on Jan. 8, 2024, and GB Patent Application No. 2316995.6, filed on Nov. 6, 2023, which are hereby incorporated by reference in their entirety.

### FIELD OF THE DISCLOSURE

The present disclosure relates to novel pharmaceutical formulations, including solid oral dosage forms comprising the non-psychoactive cannabinoid cannabidiol (CBD). The disclosure further relates to methods of manufacture of the pharmaceutical formulations and the use of the formulations to treat diseases and conditions.

### BACKGROUND

Cannabinoids in the form of botanical, plant-based cannabis, have been used in medicine for millennia, with evidence of its first use around 400AD. Cannabinoids are highly lipophilic and as such present a problem with their formulation. Early use of cannabis as medicine involved the preparation of tinctures where the cannabis is extracted and concentrated in an alcohol which is then administered in drop form.

In recent times, cannabinoids have been more thoroughly researched and have evidenced efficacy as medicaments, which has necessitated finding more effective ways of drug delivery.

There are currently four approved drugs which contain cannabinoids. Dronabinol comprises synthetically produced tetrahydrocannabinol (THC) which is formulated in sesame oil and administered orally as capsules. Dronabinol is indicated as an appetite stimulant in patients with HIV/AIDS and cancer and it is additionally approved to treat chemotherapy-induced nausea and vomiting.

Another drug derived from THC is nabilone which comprises an analogue of THC. Nabilone is formulated with povidone and corn starch and again is delivered orally as capsules. Nabilone is indicated for similar treatments as dronabinol.

The medication nabiximols is a botanical extract comprising a high-THC and a high-CBD extract in approximately equal amounts. Due to the highly lipophilic nature of the plant extract the drug is formulated with ethanol, propylene glycol and peppermint flavouring and is delivered as an oromucosal spray which is sprayed into the cheek of the user. Nabiximols was approved by the Medicines and Healthcare products Regulatory Agency (MHRA) in 2010 to treat neuropathic pain, spasticity, and overactive bladder in patients with multiple sclerosis.

The cannabinoid CBD is approved as the medication Epidiolex for the treatment of seizures associated with the rare epilepsy syndromes Dravet syndrome, Lennox-Gastaut syndrome and tuberous sclerosis complex. The CBD is formulated as 100 mg/ml in sesame seed oil and further comprises the sweetener sucralose, strawberry flavouring and up to 10% v/v ethanol which is administered as an oral solution. Epidiolex is dosed at 5 mg/kg/day for a week, titrating up to a maximum dose of 25 mg/kg/day, therefore meaning a 50 kg child would be required to take 12.6 ml of Epidiolex per day. Furthermore, adults taking Epidiolex would need to take a considerable amount more than a child, given that the doses are calculated based on weight. In this situation the adult would be taking a large volume of oil on a daily basis which can lead to pernicious side effects.

Cannabinoids are highly lipophilic and as such cause difficulties during their formulation, particularly where a substantial dose of cannabinoids are required, for example in the case of CBD. Due to the poor water solubility of cannabinoids, they are often formulated in oils or alcohols or a combination of the two which leads to problems particularly where large doses are required. A high intake of oils can cause gastrointestinal problems such as vomiting, or diarrhoea and are contraindicated in subjects with an allergy to the nut/oil. Also, a high alcohol content is contraindicated in children and adults with alcohol sensitivities.

The oral bioavailability of CBD is approximately 6% in humans due to extensive first-pass metabolism, while its bioavailability via inhalation is 11 to 45% (mean 31%). Following administration to healthy subjects of a single 400 mg oral dose encapsulated in gelatine capsules, CBD was found to be rapidly absorbed, with mean peak plasma concentrations of 114 to 181 ng/ml being attained at about 2.5 to 3 hours at steady state (Devinsky et al., 2014).

The patent application WO 2015/184127 describes several cannabinoid oral formulations such as an alcohol-free formulation where a cannabinoid is formulated in a mixture of polyethylene glycol and propylene glycol. The application additionally describes formulations where the cannabinoid is dissolved in lipids and another comprising alcohol.

The patent applications WO 2021/081138 and WO 2021/081140 describe compositions and methods of preparing said compositions whereby a lipophilic active pharmaceutical ingredient (API) is encapsulated using cyclodextrins. The compositions produced are described as having a 200% increase in bioavailability compared to non-cyclodextrin-encapsulated APIs and utilise highly purified, 99.9% pure API.

The patent application EP4252745 describes cannabinoid formulations with varying components. Cannabidiol (10 or 20%), a combination of three different cyclodextrins (30 or 55%) and non-specific poloxamers (26 or 35%), plus 8 other excipients including PEG, EDTA, and citric acid which are used to produce a formulation. The data presented in the application demonstrates that they are able to produce an effervescent tablet with either 15 mg or 30 mg of CBD.

Another cannabinoid formulation is described in CN112891310. Here a full spectrum cannabinoid oil which comprises 50-83% CBD. This is used to prepare a formulation comprising an emulsifier (which can be poloxamer-188), and a carrier (which can be a cyclodextrin). Example 1 details a combination of poloxamer-188 and HP-β-CD, the CBD content of the formulation is 6.05%.

The application US2023/0000770 describes a method of preparing a cannabinoid nano-micelle powder. The cannabinoid CBD is present with an amphiphilic polymer (which can be a poloxamer) and a freeze-drying agent (which can be a cyclodextrin, including HP-β-CD). Example 1, Table 1 details CBD (10-60%), poloxamer-188 (0, 10 or 50%), HP-β-CD (0 or 5%) and up to 17 additional excipients. The effervescent tablets prepared in Example 4 comprise either 20, 40 or 60 mg of CBD.

The application US2021/0393784 describes a method for treating pain comprising injecting a subject with a cannabinoid solubilized in SBE-7-β-cyclodextrin and optionally a solubility enhancer (which can be poloxamer-188). The cannabinoid can be CBD and is present at 1-5 mg/ml.

The buccal or sublingual dosage form described in US2011/0028431 comprises a non-ionic polymeric solubility enhancer (which can be a poloxamer), a pharmaceutically active agent (which is a water-soluble complex of a cannabinoid and a cyclodextrin) in addition to a mucoadhesive polymer, a disintegrant and a filler. The cannabinoid-cyclodextrin complex is present at 5-65%. The amount of cannabinoid used to make the CB-CD complex are between 0.01-10 (THC, Example 1) and 1-20% (CBD, Example 2). Therefore, the maximum percentage of CBD in the final formulation will be 13% (20% CBD in 65% CB-CD complex).

When cannabinoids are administered via the oral route the bioavailability is generally very poor as most of the cannabinoids are lost to primary metabolism called the first-pass effect, whereby the active cannabinoid is quickly metabolised by the liver into inactive metabolites.

The present disclosure provides novel oral cannabinoid formulations which have been shown to produce a higher bioavailability than currently available formulations without side effects. Such formulations solve the problems associated with administering high doses of cannabinoids in oil and/or alcoholic solutions.

The novel formulation of the disclosure provides the ability to dose the cannabinoid in a solid oral dosage form such as a pill, capsule or tablet which enables a more pleasant experience for the patient and greater patient compliance.

Furthermore, as the overview of the background prior art above demonstrates the amount of cannabinoid that can be solubilised into a solid oral dosage form is reasonably low. In consequence this means that patients that need to take a high dose of a cannabinoid, for example, CBD used to treat epilepsy is dosed at 20-25 mg/kg/day which for a 70 kg person would amount to 1,400-1,750 mg of CBD per day. The pill burden for these doses would therefore mean patients needing to take greater than 10 capsules/tablets per day. This is generally unacceptable for a medication that needs to be taken on a daily basis.

The present application enables a higher loading of CBD per capsule enabling a lower pill burden for patients.

### BRIEF SUMMARY OF THE DISCLOSURE

In accordance with a first aspect of the present disclosure there is provided a solid, oral pharmaceutical dosage form comprising cannabidiol, hydroxypropyl-β-cyclodextrin (HPBCD), poloxamer-188, and poloxamer-407, wherein the cannabidiol is present in an amount from 100-300 mg per unit dose.

In one embodiment the cannabidiol is present in an amount of between 20% (w/w) to 50% (w/w) of the total composition.

In a further embodiment the hydroxypropyl-β-cyclodextrin (HPBCD) is present in an amount of between 15% (w/w) and 35% (w/w).

In a further embodiment the poloxamer-188 is present in an amount of between 20% (w/w) to 40% (w/w).

In a further embodiment the poloxamer-407 is present in an amount of between 5% (w/w) to 15% (w/w).

In one embodiment the formulation further comprises one or more pharmaceutically acceptable emulsifiers and/or surfactants.

In a further embodiment the pharmaceutically acceptable emulsifier and/or surfactant is taken from the group consisting of: lecithin, glyceryl monostearate, methylcellulose, sodium lauryl sulfate, sodium oleate, sorbitan monopalmitate, sorbitan monostearate, sorbitan tristearate, tragacanth, triethanolamine oleate, polyethylene sorbitan monolaurate, polyethylene glycol (PEG), macrogol 3350, macrogol 4000, macrogol 6000, detergents, polysorbate 80 (polyoxyethylene sorbitan monooleate), polysorbate 20 (polyoxyethylene sorbitan monolaurate), cetearyl glucoside, polyglucosides, sorbitan monooleate (Span 80), sorbitan monolaurate (Span 20), polyoxyethylene monostearate (Myrj 45), polyoxyethylene vegetable oil (Emulphor), cetyl piridinium chloride, polysaccharides gums, xanthan gums, tragacanth, gum arabica, and acacia.

In one embodiment the pharmaceutically acceptable emulsifier and/or surfactant is present in an amount of between 0.1% (w/w) to 35% (w/w) of the total composition.

In a further embodiment the solid, oral pharmaceutical dosage is formulated for administration as a dosage form selected from the group consisting of: tablet, pill, pellet, capsule, powder, lozenge, granule, and sustained-release formulation.

In a further embodiment the solid, oral pharmaceutical dosage is formulated for administration before food, with food or after food.

In one embodiment the cannabidiol is present in an amount of about 250 mg per unit dose.

In a further embodiment the dosage form produces an AUC₀₋ₜ which is between 80% and 125% of the AUC₀₋ₜ of a reference compound Epidiolex® following administration of the dosage form to a subject.

In a further embodiment the dosage form produces a Cₘₐₓ which is between 80% and 125% of the Cₘₐₓ of a reference compound Epidiolex® following administration of the dosage form to a subject.

In a further embodiment there is provided a method of treatment comprising administering to a subject a therapeutically effective amount of the solid oral dosage form.

In a further embodiment there is provided a process for preparing the solid, oral pharmaceutical dosage form of the disclosure comprising the steps of:
a) Mixing the cannabidiol, hydroxypropyl-β-cyclodextrin (HPBCD), poloxamer-188, and poloxamer-407 under supercritical CO₂ conditions of 2,000-3,000 psi and 30-50° C. for 20 to 60 minutes; b) depressurising the reactor and collecting the material; heating the material collected in step (b) to 40-50° C.; and filling capsules with material whilst warm using an injection filling technique.

The following paragraphs are included with reference to earlier priority applications:
1. A pharmaceutical composition comprising one or more cannabinoids, one or more cyclodextrins or cyclodextrin derivatives, and one or more poloxamers and optionally one or more pharmaceutically acceptable emulsifiers and/or surfactants.
2. A pharmaceutical composition according to claim 1, wherein the said composition exhibits an AUC₀₋ₜ which is between 80% and 125% of the reference compound following administration of the composition to a subject.
3. A pharmaceutical composition according to claim 1 or claim 2, wherein the said composition exhibits a Cₘₐₓ which is between 80% and 125% of the reference compound following administration of the composition to a subject.
4. A pharmaceutical composition according to any of the preceding claims, wherein the one or more cannabinoids are taken from the group consisting of: cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCVA).
5. A pharmaceutical composition according to claim 4 wherein the one or more cannabinoids are present in an amount of between 15% (w/w) to 25% (w/w) of the total composition.
6. A pharmaceutical composition according to claim 4 or claim 5 wherein the one or more cannabinoid is cannabidiol (CBD).
7. A pharmaceutical composition according to claim 1 wherein the one or more cyclodextrins or cyclodextrin derivatives are taken from the group consisting of: α-cyclodextrin, β-cyclodextrin, γ-cyclodextrin, α-cyclodextrin exadeacetate (AACD), β-cyclodextrin heneicosaacetate (ABCD), γ-cyclodextrin octadeacetate (AGCD), hydroxypropyl-α-cyclodextrin (HPαCD), hydroxypropyl-β-cyclodextrin (HPBCD), hydroxypropyl-γ-cyclodextrin (HPγCD), methyl-α-cyclodextrin (MαCD), methyl-β-cyclodextrin (MβCD), methyl-γ-cyclodextrin (MγCD), sulfobutylether-α-cyclodextrin (SBEαCD), sulfobutylether-β-cyclodextrin (SBEβCD) and sulfobutylether-γ-cyclodextrin (SBEγCD).
8. A pharmaceutical composition according to claim 7 wherein the one or more cyclodextrins or cyclodextrin derivatives are present in an amount of between 25% (w/w) to 50% (w/w) of the total composition.
9. A pharmaceutical composition according to claim 7 or claim 8 wherein the one or more cyclodextrins or cyclodextrin derivatives is hydroxypropyl-β-cyclodextrin (HPBCD).
10. A pharmaceutical composition according to claim 1 wherein the one or more poloxamers are taken from the group consisting of: poloxamer-182, poloxamer-183, poloxamer-184, poloxamer-185, poloxamer-188, poloxamer-212, poloxamer-215, poloxamer-217, poloxamer-234, poloxamer-235, poloxamer-237, poloxamer-238, poloxamer-288, poloxamer-333, poloxamer-334, poloxamer-335, poloxamer-338, poloxamer-402, poloxamer-403, and poloxamer-407.
11. A pharmaceutical composition according to claim 10 wherein the one or more poloxamers are present in an amount of between 10% (w/w) to 70% (w/w) of the total composition.
12. A pharmaceutical composition according to claim 10 or claim 11 wherein the one or more poloxamers are poloxamer-188 and/or poloxamer-407.
13. A pharmaceutical composition according to claim 1 wherein the one or more pharmaceutically acceptable emulsifiers and/or surfactants are taken from the group consisting of: lecithin, glyceryl monostearate, methylcellulose, sodium lauryl sulfate, sodium oleate, sorbitan monopalmitate, sorbitan monostearate, sorbitan tristearate, tragacanth, triethanolamine oleate, polyethylene sorbitan monolaurate, polyethylene glycol (PEG), macrogol 3350, macrogol 4000, macrogol 6000, detergents, polysorbate 80 (polyoxyethylene sorbitan monooleate), polysorbate 20 (polyoxyethylene sorbitan monolaurate), cetearyl glucoside, polyglucosides, sorbitan monooleate (Span 80), sorbitan monolaurate (Span 20), polyoxyethylene monostearate (Myrj 45), polyoxyethylene vegetable oil (Emulphor), cetyl piridinium chloride, polysaccharides gums, xanthan gums, tragacanth, gum arabica, acacia.
14. A pharmaceutical composition according to claim 13 wherein the one or more pharmaceutically acceptable emulsifiers and/or surfactants are present in an amount of between 0% (w/w) to 35% (w/w) of the total composition.
15. A pharmaceutical composition according to claim 13 or claim 14 wherein the one or more pharmaceutically acceptable emulsifiers and/or surfactants are macrogol 6000 and/or polysorbate 80.
16. A pharmaceutical composition according to any of the preceding claims comprising cannabidiol, hydroxypropyl-β-cyclodextrin (HPBCD), poloxamer-188, poloxamer-407 and optionally macrogol 6000 and polysorbate 80.
17. A pharmaceutical composition according to claim 16 wherein the cannabidiol is present at an amount of between 15% (w/w) to 25% (w/w) of the total composition, the hydroxypropyl-β-cyclodextrin (HPBCD) is present at an amount of between 25% (w/w) to 50% (w/w) of the total composition, the poloxamer-188 and poloxamer-407 are present at an amount of between 10% (w/w) to 70% (w/w) of the total composition and the macrogol 6000 and polysorbate 80 are present at an amount of between 0% (w/w) to 35% (w/w) of the total composition.
18. A pharmaceutical composition according to any of the preceding claims wherein the subject is a mammal.
19. A pharmaceutical composition according to any of the preceding claims wherein the subject is a human.
20. A pharmaceutical composition according to any of the preceding claims wherein the composition is in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, a liquid, a lozenge, a fast disintegrating tablet, a lyophilized preparation, a film, a spray (including a nasal spray, an oral spray, or a topical spray), or a mucoadhesive.
21. A pharmaceutical composition according to any of the preceding claims wherein the composition is formulated for administration as a dosage form selected from the group consisting of: tablets, pills, pellets, capsules, powders, lozenges, granules, solutions, suspensions, emulsions, syrups, elixirs, sustained-release formulations, aerosols, and sprays.
22. A pharmaceutical composition according to any of the preceding claims wherein the composition is administered before food.
23. A pharmaceutical composition according to any of the preceding claims wherein the composition is administered after food.
24. A pharmaceutical composition according to any of the preceding claims wherein the composition is formulated as a capsule.
25. A pharmaceutical composition according claim 24 wherein the composition comprises cannabidiol.
26. A pharmaceutical composition according to claim 25 wherein the cannabidiol is present in an amount of from 127.5 mg to 172.5 mg of cannabidiol per capsule.
27. A pharmaceutical composition according to claim 26 wherein the cannabidiol is present in an amount of about 150 mg of cannabidiol per capsule.
28. A method of treatment comprising administering to a subject a therapeutically effective amount of the composition according to any of the preceding claims.

### BRIEF SUMMARY OF THE DRAWINGS

The present disclosure is described with reference to the figures listed below:

FIG. 1 details the mean CBD plasma concentration (0-24 h) of Epidiolex (red diamond), Formulation T1 (blue circle) and Formulation T2 (green triangle).

FIG. 2 details the mean 7-OH-CBD plasma concentration (0-96 h) of Epidiolex (red diamond), Formulation T1 (blue circle) and Formulation T2 (green triangle).

FIG. 3 details the mean 7-COOH-CBD plasma concentration (0-96 h) of Epidiolex (red diamond), Formulation T1 (blue circle) and Formulation T2 (green triangle).

FIG. 4 details the mean CBD plasma concentration (0-24 h) of Formulation T1 under fed conditions (blue circle) and Formulation T1 under fasted conditions (red diamond).

FIG. 5 details the mean 7-OH-CBD plasma concentration (0-96 h) of Formulation T1 under fed conditions (blue circle) and Formulation T1 under fasted conditions (red diamond).

FIG. 6 details the mean 7-COOH-CBD plasma concentration (0-96 h) of Formulation T1 under fed conditions (blue circle) and Formulation T1 under fasted conditions (red diamond).

### DEFINITIONS

“About” refers to all values having substantially the same effect, or providing substantially the same result, as the reference value. Thus, the range encompassed by the term “about” will vary depending on the context in which the term is used, for instance the parameter that the reference value is associated with. Thus, depending on context, “about” can mean, for example: +0.10%; +0.25%; +0.5%; +1.0%; +2.5%; +5.0%; or +10.0%. All recitations of a reference value preceded by the term “about” are intended to also be a recitation of the reference value alone.

The term “about” when used in respect to pharmacokinetic parameters, such as AUC; AUC₀₋ₜ; AUC_{0-inf}; Cₘₐₓ; Tₘₐₓ means from 80% to 125% of the reference parameter.

“Absorption profile” refers to the rate and extent of exposure of a drug by data analysis of the AUC and/or Cₘₐₓ including the curves thereof.

“Administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, and transdermal. In particular, the administration method can be oral administration.

“Bioequivalence” means the absence of a significant difference in the rate and extent to which the active agent or surrogate marker for the active agent in pharmaceutical equivalents or pharmaceutical alternatives becomes available at the site of action when administered in an appropriately designed study. The 90% Cl limits for a ratio of the geometric mean of logarithmic transformed Cₘₐₓ for the two products or methods can have a wider acceptance range when justified by safety and efficacy considerations.

“Co-administration” refers to administration of two or more different active agents together in a coordinated manner. Co-administration includes administration of two or more different active agents simultaneously, sequentially, or separately. Thus, “co-administration” includes administration in the same or different dosage forms, concurrent administration, as well as administration that is not concurrent, such as administration of a first active agent followed or alternated with administration of a second active agent as part of a coordinated plan for treatment.

A “composition” is a collection of materials containing the specified components. One or more dosage forms may constitute a composition, so long as those dosage forms are associated and designed for use together.

“Enteric polymer” refers to a polymer that is poorly soluble in aqueous medium at a pH of about 4.5 or less but becomes soluble in aqueous medium at a pH of greater than about 5. For example, an enteric polymer is poorly soluble in gastric juice but is soluble in the lower GI tract environment.

“Pharmaceutical composition” refers to a formulation of a compound of the disclosure, such as cannabidiol, and a medium generally accepted in the art for the delivery of the biologically active compound to mammals, e.g., humans. Such a medium includes all pharmaceutically acceptable carriers, diluents or excipients therefor. The pharmaceutical composition may be in various dosage forms or contain one or more unit dose formulations.

“Pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

A “reference composition of cannabidiol” (reference composition) is a composition comprising cannabidiol that an AUC₀₋ₜ in the fasted state that is about 35% or more lower than the AUC₀₋ₜ in the fed state; and/or has an intra-subject variability of about 30% or greater.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminium, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In embodiments, the pharmaceutically acceptable base addition salt is chosen from ammonium, potassium, sodium, calcium, and magnesium salts.

“Solvate” means a complex formed by solvation (the combination of solvent molecules with molecules or ions of the active agent of the present disclosure), or an aggregate that consists of a solute ion or molecule (the active agent of the present disclosure) with one or more solvent molecules. For example, a solvate where the solvent molecule or molecules are water is called a hydrate. Hydrates are particularly contemplated as solvates of the materials described herein.

“Solid dispersion” relates to a solid system comprising a nearly homogeneous or homogeneous dispersion of an active ingredient in an inert carrier or matrix.

“Substantially similar to” means having a great extent or degree of likeness to the reference item, term, quantity, etc.

“Prodrug” refers to a precursor of the active agent wherein the precursor itself may or may not be pharmaceutically active but, upon administration, will be converted, either metabolically or otherwise, into the active agent or drug of interest. For example, prodrug includes an ester or an ether form of an active agent.

“Therapeutically effective amount” or “effective amount” refers the amount of a pharmaceutically active agent, that, when administered to a patient for treating a disease according to the dosing regimen as described herein, is sufficient to affect such treatment for the disease. The “therapeutically effective amount” will vary depending on the disease and its severity, and the age, weight, and other conditions of the patient to be treated.

A composition or dosage form is “therapeutically equivalent” to a reference composition or dosage form if it has a therapeutic effect that is substantially like the therapeutic effect of the reference composition or dosage form, for example, therapeutically equivalent dosage forms can have substantially similar efficacy towards a particular disease or condition when administered over a substantially similar time period.

“Patient” or “subject” refers to a mammal, e.g., a human, in need of medical treatment.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

“Cyclodextrin/s” or “CD” are cyclic oligosaccharides consisting of alpha 1-4 linked D-glucopyranose units. The number of glucopyranosides which are linked refers to the category of cyclodextrin. The three main types of cyclodextrin are alpha (α) cyclodextrins have 6 glucose subunits, beta (β) cyclodextrins have 7 glucose subunits, and gamma (γ) cyclodextrins have 8 glucose subunits are the most commonly used natural cyclodextrins although larger cyclodextrins have been reported. Cyclodextrins can be used as pharmaceutical excipients and are deemed generally recognised as safe (GRAS) by the FDA for human use. In the context of the present disclosure the term cyclodextrin refers to any in known cyclodextrin.

“Cyclodextrin derivatives” the hydroxyl group of cyclodextrins can be modified producing cyclodextrin derivatives. For example, propylene oxide produces hydroxypropyl-cyclodextrin derivatives. Cyclodextrin derivatives often have superior properties for example, β-cyclodextrin and MβCD remove cholesterol from cultured cells, however the methylated form MβCD was found to be more efficient than β-cyclodextrin. Certain cyclodextrins are approved by the FDA and EMA for inclusion in drugs. For example, 2-Hydroxypropyl-β-cyclodextrin (HP-β-CD) is used in the antifungal API itraconazole, in both intravenous and oral solutions. In the context of the present disclosure the term cyclodextrin derivative refers to any in known cyclodextrin derivative.

“Poloxamer/s” are non-ionic triblock copolymers composed of a central hydrophobic chain of polyoxypropylene (poly(propylene oxide)) which has a hydrophilic chain of polyoxyethylene (poly(ethylene oxide)) on either side. Many different poloxamers can be formed by using various lengths of the polymer blocks resulting in slightly different properties. Poloxamers have surfactant properties and are often used to increase the water solubility of pharmaceuticals. In the context of the present disclosure the term poloxamer refers to any in known poloxamer.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

The following Examples describe novel oral cannabinoid formulations. These formulations have been tested and were found to produce a higher bioavailability than currently available formulations without side effects. Such formulations solve the problems associated with administering high doses of cannabinoids in oil and/or alcoholic solutions. The novel formulation of the disclosure provides the ability to dose the cannabinoid in a solid oral dosage form such as a pill, capsule or tablet which enables a more pleasant experience for the patient and greater patient compliance.

### Active Pharmaceutical Ingredient (API)

The API used for the present application is one or more cannabinoid selected from those disclosed in Handbook of Cannabis, Roger Pertwee, Chapter 1, pages 3 to 15.

The preferred cannabinoids of the disclosure are cannabidiol (CBD) and/or tetrahydrocannabinol (THC).

It is preferred that the cannabinoid is present in an amount of from about 5 to 80 wt %, based on the total composition, preferably from about 10 to 50 wt %, more preferably from about 15 to 25 wt %.

Preferably, the cannabinoid is synthetic or highly purified from its natural source (for example, plant derived recrystallized form). When a highly purified source is used, it is purified such that the cannabinoid is present at greater than 95% of the total extract (w/w). Use of a synthetic or highly purified cannabinoid is advantageous because these contain relatively low amounts of wax. This assists in prevention of the formation of an oily formulation, increasing physical stability of the formulation.

The unit dose of cannabinoid in the oral pharmaceutical formulation may be in the range of from 0.001 to 1000 mg, preferably 1 to 500 mg, more preferably 150 to 350 mg. For example, it is envisaged that, when in tablet or capsule unit dose form, the amount of cannabinoid present may be about 0.5, 1, 2, 5, 10, 25, 50, 100, 125, 150, 175, 200, 210, 215, 220, 225, 230, 235, 240, 245, 250, 255, 260, 265, 270, 275, 280, 285, 290, 295, 300, 325, 350, 375, 400, 450, 500, 550, 600, 650, 700, 750, 800, 850, 900, 950, 1000 mg.

### Preparation of Test Articles

The procedure for preparing the formulations of the disclosure were as follows:

The ingredients were weighed using a calibrated balance and transferred to a clean and dry wide mouth glass jar.

The glass jar was closed with a SureTight lid and shaken well for 2-3 minutes to mix and seal it with parafilm.

The powder mix was prepared in a 150 ml stainless-steel high-pressure reactor. All ingredients were added, and the reactor was sealed prior to the addition of the solvent. CO₂ was added and pressure and temperature were increased to reach the supercritical phase. Reactor was held between 2000 and 3000 psi with a temperature between 3° and 50° C. and the contents were vigorously mixed for between 20 and 60 minutes. The reactor was depressurized over 1 hour and the resultant inclusion complex powder was collected.

It should be noted that the use of supercritical CO₂ was solely for the preparation of the formulation of the disclosure, and it was not used for extraction of cannabinoids or other components.

The formulation could alternatively be prepared using the process described in WO2021/081138 which is incorporated by reference.

### EXAMPLES

### Example 1: Evaluation of the Pharmacokinetics of Cannabidiol (CBD) in Plasma Following Oral Administration of Two CBD Formulations to Male Sprague-Dawley Rats

The plasma pharmacokinetics (PK) of 10 mg/kg cannabidiol (CBD) were measured following oral administration to male Sprague-Dawley rats of two different CBD formulations: CBD-1 and CBD MCT oil tincture. Catheters were placed in the carotid artery of 8 rats for blood collection. Each formulation was administered to a group of 4 catheterized rats and blood plasma was serially collected at 12 time-points over 24 hours following dosing.

The mean maximum CBD plasma concentrations of the two formulations were calculated along with the times in which these were achieved.

### Materials and Methods

Eight male Sprague-Dawley rate (250 g-325 g) (Charles River) were used for the study. The animals were acclimatised to their environment for a minimum of 5 days prior to surgery.

Catheters were implanted in the carotid artery in all animals for serial blood collection and blood sample volume replacement. Catheters were implanted at least one day prior to the start of the dosing procedures.

Animals were separated into two treatment groups, four per group, and were dosed by oral gavage with test article.

Blood samples were taken at time points: 3, 5, 10, 20, 30, 45, 60, 90, 120, 240, 480, and 1440 minutes and the samples were tested using a quantified bioanalytical method for the amount of CBD.

To determine if statistically significant differences in CBD plasma exposure (Cₘₐₓ and AUC) existed between the 3 formulations, one-way ANOVA was performed with Tukey's multiple comparisons test using GraphPad Prism® 9.1 software. A P value of <0.05 was considered significant.

### Test Articles

Two different CBD-containing test articles were used in this study. CBD-1 was formulated with cyclodextrin, and CBD-2 was formulated with a medium chain triglyceride oil. Details of the test articles are described in Table 1 below.

TABLE 1
Test article details

| | | THC Dose | Inactive |
|---|---|---|---|
| Test | Route of | (mg) per | Ingredients/ |
| Article | Administration | Administration | Excipients |
| CBD-1 | oral gavage | 10 | Poloxamer-407, |
| | | | α-cyclodextrin |
| CBD-2 | oral gavage | 10 | Medium chain |
| | | | triglyceride |

### Preparation of Test Article:

The procedure for preparing the CBD-1 was as described previously.

TABLE 2
Composition of CBD-1 formulation

| | | Quantity |
|---|---|---|
| | Composition | per batch |
| Ingredient | (% w/w) | (grams) |
| Cannabidiol | 16.7% | 2 |
| Poloxamer-407 | 66.6% | 8 |
| α-cyclodextrin | 16.7% | 2 |
| TOTAL | 100% | 12 |

### Results

The in vivo PK parameters for CBD following 10 mg/kg per os (“p.o.” i.e. orally or by mouth) administration of the two different formulations are summarised in Table 3, and pharmacokinetics (“PK”) plasma parameters comparisons (fold change) between the two formulations are summarised in Table 4.

TABLE 3
Summary of mean PK parameters

| | Formulation | |
|---|---|---|
| Parameter | CBD-1 | CBD in MCT |
| Administered dose (mg/kg)^{a} | 11.7 | 12.7 |
| tₘₐₓ (min) | 52.5 ± 8.66 | 420 ± 120^{b} |
| Cₘₐₓ (ng/ml) | 195 ± 23.1 | 14.9 ± 14.6^{b} |
| Cₘₐₓ/Dose (kg*ng/ml/mg) | 16.7 ± 1.98 | 1.17 ± 1.15^{b} |
| Apparent t_{1/2} (min) | 295 ± 27.3 | n/c |
| AUC₀₋ₜₗₐₛₜ (min*ng/ml) | 25700 ± 5200 | 6710 ± 6520 |
| AUC₀₋ₜₗₐₛₜ/Dose (min*kg*ng/ml/mg) | 2200 ± 445 | 528 ± 513^{b} |

Key:
^{a}administered dose = measured dosing solution concentration multiplied by dose volume
^{b}significantly different from CBD-1 group (p < 0.05)
n/c—not calculable as the terminal phase not well defined

As can be observed the CBD-1 formulation had a significantly faster onset than the CBD in MCT with a tₘₐₓ of 52.5 minutes versus 7 hours for the CBD in MCT oil. The Cₘₐₓ was also significantly more favourable in the CBD-1 formulation with a mean of 195 ng/ml compared to 15 for the CBD in MCT.

Table 4 below details the PK parameters in comparison to each other.

TABLE 4
CBD PK plasma parameters comparison (fold change)
between CBD-1 formulation versus CBD MCT tincture

| | CBD-1 vs |
|---|---|
| Parameter | CBD in MCT |
| tₘₐₓ (min) | 0.125 |
| Cₘₐₓ/Dose (kg*ng/ml/mg) | 14.2 |
| AUC₀₋ₜₗₐₛₜ/Dose (min*kg*ng/ml/mg) | 4.16 |

The dose-normalised maximum plasma concentration (Cₘₐₓ/Dose) attained following administration of the CBD-1 formulation was 16.7 kg*ng/ml/mg and was significantly higher (14-fold P<0.0001) than that following administration of the CBD MCT tincture (1.17 kg*ng/ml/mg).

Administration of CBD-1 formulation resulted in a significantly higher (4-fold; P=0.0027) dose-normalized CBD exposure (2200 min*kg*ng/mL/mg) when compared to CBD MCT tincture administration (528 min*kg*ng/mL/mg)

### Conclusion

The novel CBD-1 formulation of the disclosure in which the CBD was solubilized using α-cyclodextrin and poloxamer-407 has been shown to exhibit favourable PK parameters when administered to rats. The substantially faster tₘₐₓ denotes that this formulation provides a faster onset than CBD formulated in MCT oil. In addition, the higher Cₘₐₓ demonstrated by the novel formulation provides the ability to administer lower doses to achieve the same effect in comparison to the other two formulations tested. This provides many advantages including a lower cost of goods and a lower side effect profile.

### Example 2: Evaluation of the Pharmacokinetics of Cannabidiol (CBD) in Plasma Following Oral Administration of Four CBD Formulations to Male Sprague-Dawley Rats

The plasma pharmacokinetics (PK) of 10 mg/kg cannabidiol (CBD) following oral administration of four different CBD formulations; CBD-1, CBD-2, CBD-3 and CBD-4 to male Sprague-Dawley rats. Catheters were placed in the carotid artery of 16 rats for blood collection. Each formulation was administered to a group of 4 catheterized rats and blood plasma was serially collected at 12 time-points over 24 hours following dosing.

The mean maximum CBD plasma concentrations of the four formulations were calculated along with the times in which these were achieved.

### Materials and Methods

Sixteen male Sprague-Dawley rate (250 g-325 g) (Charles River) were used for the study. The animals were acclimatised to their environment for a minimum of 5 days prior to surgery.

Catheters were implanted in the carotid artery in all animals for serial blood collection and blood sample volume replacement. Catheters were implanted at least one day prior to the start of the dosing procedures.

Animals were separated into two treatment groups, four per group, and were dosed by oral gavage with test article.

Blood samples were taken at time points: 3, 5, 10, 20, 30, 45, 60, 90, 120, 240, 480, and 1440 minutes and the samples were tested using a quantified bioanalytical method for the amount of CBD.

To determine if statistically significant differences in CBD plasma exposure (Cₘₐₓ and AUC) existed between the 3 formulations, one-way ANOVA was performed with Tukey's multiple comparisons test using GraphPad Prism® 9.1 software. A P value of <0.05 was considered significant.

### Test Articles

Four different CBD-containing test articles were used in this study. CBD-1 was formulated with α-cyclodextrin and poloxamer-407 and was the same formulation used in Example 1. The remainder of the formulations used hydroxypropyl-β-cyclodextrin (HPBCD) along with various additional excipients as described in Table 5 below.

TABLE 5
Test article details

| | | THC Dose (mg) | Inactive |
|---|---|---|---|
| Test | Route of | per | Ingredients/ |
| Article | Administration | Administration | Excipients |
| CBD-1 | oral gavage | 10 | α-cyclodextrin |
| | | | Poloxamer-407 |
| CBD-2 | oral gavage | 10 | HPβCD |
| | | | Poloxamer-407 |
| | | | Poloxamer-188 |
| CBD-3 | oral gavage | 10 | HPβCD |
| | | | Poloxamer-407 |
| | | | Poloxamer-188 |
| | | | Polysorbate 80 |
| | | | PEG-6000 |
| CBD-4 | oral gavage | 10 | HPβCD |
| | | | Poloxamer-407 |
| | | | Poloxamer-188 |
| | | | Polysorbate 80 |
| | | | PEG-1500 |

### Preparation of Test Article:

The procedure for preparing the CBD formulations using the ingredients detailed in Tables 6 to 9 were as previously described.

TABLE 6
Composition of CBD-1 formulation

| | | Quantity |
|---|---|---|
| | Composition | per batch |
| Ingredient | (% w/w) | (grams) |
| Cannabidiol | 16.7% | 2 |
| Poloxamer-407 | 66.6% | 8 |
| α-cyclodextrin | 16.7% | 2 |
| TOTAL | 100% | 12 |

TABLE 7
Composition of CBD-2 formulation

| | | Quantity |
|---|---|---|
| | Composition | per batch |
| Ingredient | (% w/w) | (grams) |
| Cannabidiol | 22.2% | 2 |
| Poloxamer-407 | 11.1% | 1 |
| Poloxamer-188 | 33.3% | 3 |
| hydroxypropyl-β- | 33.3% | 3 |
| cyclodextrin | | |
| TOTAL | 100% | 9 |

TABLE 8
Composition of CBD-3 formulation

| | | Quantity |
|---|---|---|
| | Composition | per batch |
| Ingredient | (% w/w) | (grams) |
| Cannabidiol | 16.7% | 2 |
| Poloxamer-407 | 4.2% | 0.5 |
| Poloxamer-188 | 12.5% | 1.5 |
| hydroxypropyl-β- | 33.3% | 4 |
| cyclodextrin | | |
| Polysorbate 80 | 16.7% | 2 |
| PEG-6000 | 16.7% | 2 |
| TOTAL | 100% | 12 |

TABLE 9
Composition of CBD-4 formulation

| | | Quantity |
|---|---|---|
| | Composition | per batch |
| Ingredient | (% w/w) | (grams) |
| Cannabidiol | 16.7% | 2 |
| Poloxamer-407 | 4.2% | 0.5 |
| Poloxamer-188 | 12.5% | 1.5 |
| hydroxypropyl-β- | 33.3% | 4 |
| cyclodextrin | | |
| Polysorbate 80 | 16.7% | 2 |
| PEG-1500 | 16.7% | 2 |
| TOTAL | 100% | 12 |

### Results

The in vivo PK parameters for CBD following 10 mg/kg p.o. administration of four different formulations are summarised in Table 10, and PK plasma parameters comparisons (fold change) between the four formulations are summarised in Table 11.

TABLE 10
Summary of mean PK parameters

| | Formulation | | | |
|---|---|---|---|---|
| Parameter | CBD-1 | CBD-2 | CBD-3 | CBD-4 |
| tₘₐₓ (min) | 41.3 ± 14.4 | 56.3 ± 7.5 | 105 ± 93.3 | 97.5 ± 99.1 |
| Cₘₐₓ (ng/ml) | 95.1 ± 57.9 | 135 ± 32.9 | 52.8 ± 23.9 | 102 ± 91.4 |
| Apparent t_{1/2} (min) | 271 ± 26.9 | 277 ± 53.8 | 251 ± 63.2 | 324 ± 28.8 |
| AUC_{0-inf} | 14200 ± 8640 | 18500 ± 1680 | 12300 ± 5340 | 14700 ± 8340 |
| (min*ng/ml) | | | | |

Key:
^{a}administered dose = measured dosing solution concentration multiplied by dose volume
^{b}significantly different from THC-1 group (p < 0.05)

As can be observed the CBD-1 formulation had a significantly faster onset than the CBD in MCT with a tₘₐₓ of 52.5 minutes versus 7 hours for the CBD in MCT oil. The Cₘₐₓ was also significantly more favourable in the CBD-1 formulation with a mean of 195 ng/ml compared to 15 for the CBD in MCT.

Table 11 below details the PK parameters in comparison to each other.

TABLE 11
CBD PK plasma parameters comparison (fold
change) between the various formulations

| | Formulation | | | |
|---|---|---|---|---|
| | CBD-2 vs | CBD-3 vs | CBD-4 vs | CBD-4 vs |
| Parameter | CBD-1 | CBD-1 | CBD-1 | CBD-3 |
| tₘₐₓ (min) | 1.36 | 2.55 | 2.36 | 0.929 |
| Cₘₐₓ (ng/ml) | 1.42 | 0.555 | 1.07 | 1.93 |
| Apparent t_{1/2} (min) | 1.02 | 0.926 | 1.20 | 1.29 |
| AUC_{0-inf} | 1.31 | 0.872 | 1.04 | 1.19 |
| (min*ng/ml) | | | | |

Following administration of the CBD-1 formulation, maximum CBD plasma concentrations (Cₘₐₓ=91 ng/mL) were achieved between 30- and 60-min post-dose (mean tₘₐₓ=41 min).

Administration of the CBD-2 formulation resulted in a Cₘₐₓ of 135 ng/ml (1.4-fold higher than formulation CBD-1) and tₘₐₓ of 56 min.

CBD levels peaked between 30- and 240-minutes following administration of both CBD-3 and CBD-4 formulations with corresponding Cₘₐₓ values of 52.8 ng/ml and 102 ng/mL (0.56 and 1.1-fold that of formulation CBD-1), respectively.

The apparent half-life (t1/2) of CBD was similar among the 8A, SP-14 and SP-24 formulations (271, 277 and 251 min, respectively), whereas the estimated CBD t1/2 was slightly longer for the P-25 formulation (324 min, Table 1).

Overall plasma exposure (AUC0-inf) of CBD was highest and least variable among animals following administration of the SP-14 formulation (18500 min*ng/ml), was comparable for the 8A and SP-25 formulations (14200 and 14700 min*ng/mL, respectively) and lowest (12300 min*ng/mL) for the SP-24 formulation (Table 1).

### Conclusion

The novel CBD-1 formulation of the disclosure in which the CBD was solubilized using α-cyclodextrin and poloxamer-407 has been shown to exhibit favourable PK parameters when administered to rats. The substantially faster tₘₐₓ denotes that this formulation provides a faster onset than CBD formulated in MCT oil (from Example 1). In addition, the higher bioavailability and Cₘₐₓ demonstrated by the novel formulation provides the ability to administer lower doses to achieve the same effect in comparison to the other two formulations tested. This provides many advantages including a lower cost of goods and a lower side effect profile.

### Example 3: Evaluation of the Pharmacokinetics of Δ9-Tetrahydrocannabinol (Δ9-THC) in Plasma Following Oral Administration of Three THC Formulations to Male Sprague-Dawley Rats

The plasma pharmacokinetics (PK) of 49-tetrahydrocannabinol (THC) was studied following oral administration of three different formulations of THC dosed at a concentration of 10 mg/kg. A cyclodextrin formulation was tested alongside a THC tincture in which the THC was dissolved in a medium chain triglyceride oil and a commercial THC emulsion.

The mean maximum THC plasma concentrations of the formulations were calculated along with the times in which these were achieved.

### Materials and Methods

Twelve male Sprague-Dawley rate (250 g-325 g) (Charles River) were used for the study. The animals were acclimatised to their environment for a minimum of 5 days prior to surgery.

Catheters were implanted in the carotid artery in all animals for serial blood collection and blood sample volume replacement. Catheters were implanted at least one day prior to the start of the dosing procedures.

Animals were separated into three treatment groups, four per group, and were dosed by oral gavage with test article.

Blood samples were taken at time points: 3, 5, 10, 20, 30, 45, 60, 90, 120, 240, 480, and 1140 minutes and the samples were tested using a quantified bioanalytical method for the amount of THC.

To determine if statistically significant differences in THC plasma exposure (Cₘₐₓ and AUC) existed between the 3 formulations, one-way ANOVA was performed with Tukey's multiple comparisons test using GraphPad Prism® 9.1 software. A P value of <0.05 was considered significant.

### Test Articles

Three different THC-containing test articles were used in this study. THC-1 was formulated with cyclodextrin, THC-2 was formulated with a medium chain triglyceride oil and THC-3 was a commercially available THC formulation. Details of the test articles is described in Table 12 below.

TABLE 12
Test article details

| | | THC Dose | |
|---|---|---|---|
| | Route of | (mg) per | Inactive |
| Test | Adminis- | Adminis- | Ingredients/ |
| Article | tration | tration | Excipients |
| THC-1 | oral gavage | 10 | Poloxamer-407, |
| | | | α-cyclodextrin |
| THC-2 | oral gavage | 10 | Medium chain |
| | | | triglyceride |
| THC-3 | oral gavage | 10 | Unknown (commercial |
| | | | formulation) |

### Preparation of Test Article:

The procedure for preparing the THC-1 formulations using the ingredients detailed in Table 13 was as previously described.

TABLE 13
Composition of THC-1 formulation

| | | Quantity |
|---|---|---|
| | Composition | per batch |
| Ingredient | (% w/w) | (grams) |
| Tetrahydrocannabinol | 16.7% | 2 |
| Poloxamer-407 | 66.6% | 8 |
| α-cyclodextrin | 16.7% | 2 |
| TOTAL | 100% | 12 |

### Results

The in vivo PK parameters for THC following 10 mg/kg p.o. administration of three different formulations are summarised in Table 14, and PK plasma parameters comparisons (fold change) between the three formulations are summarised in Table 15.

TABLE 14
Summary of mean PK parameters

| | Formulation | | |
|---|---|---|---|
| | | | Commercial |
| Parameter | THC-1 | THC in MCT | THC |
| Administered dose (mg/kg)^{a} | 9.28 | 9.63 | 8.362 |
| tₘₐₓ (min) | 35.0 ± 20.0 | 360 ± 139^{b} | 63.8 ± 39.4^{c} |
| Cₘₐₓ (ng/ml) | 65.7 ± 44.5 | 12.2 ± 5.71 | 31.9 ± 10.0 |
| Cₘₐₓ/Dose ^{a} (kg*ng/ml/mg) | 7.08 ± 4.79 | 1.27 ± 0.593 | 3.81 ± 1.20 |
| Apparent t_{1/2} (min) | 255 ± 29.5 | n/c | 278 ± 53.6 |
| AUC₀₋ₜₗₐₛₜ (min*ng/ml) | 13400 ± 3600 | 4700 ± 2660 | 9230 ± 2840 |
| AUC₀₋ₜₗₐₛₜ/Dose (min*kg*ng/ml/mg) | 1440 ± 388 | 488 ± 276^{b} | 1100 ± 340 |
| AUC_{0-inf} (min*ng/ml) | 13600 ± 3590 | n/c | 9440 ± 2860 |
| AUC_{0-inf}/Dose (min*kg*ng/ml/mg) | 1470 ± 386 | n/c | 1130 ± 342 |

Key:
^{a}administered dose = measured dosing solution concentration multiplied by dose volume
^{b}significantly different from THC-1 group (p < 0.05)
^{c}significantly different from THC in MCT group (p < 0.05)
n/c—not calculable as the terminal phase not well defined

As can be observed the THC-1 formulation had a faster onset than the other two THC formulations tested with a tₘₐₓ of 35 minutes versus over an hour for the commercial THC formulation and 6 hours for the THC in MCT oil. The Cₘₐₓ was also more favourable in the THC-1 formulation with a mean of 66 ng/ml compared to 12 and 32 for the THC in MCT and the commercial THC respectively.

A direct comparison of dose-normalized overall plasma exposure (AUC0-∞/Dose) between the different formulations was not possible, as the AUC could not be extrapolated to infinity for the THC MCT tincture group and thus a comparison of the dose normalised AUC from 0 to the last measurable concentration (tₗₐₛₜ=1440 min) was performed. Table 15 below details the PK parameters in comparison to each other.

TABLE 15
THC PK plasma parameters comparison (fold change) between THC-1
formulation versus THC MCT tincture and commercial THC

| | Formulation | |
|---|---|---|
| | THC-1 vs | THC-1 vs |
| Parameter | THC in MCT | commercial THC |
| tₘₐₓ (min) | 0.0972 | 0.549 |
| Cₘₐₓ (ng/ml) | 5.37 | 2.06 |
| Cₘₐₓ/Dose^{a} (kg*ng/ml/mg) | 5.5 | 1.86 |
| AUC₀₋ₜₗₐₛₜ (min*ng/ml) | 2.85 | 1.45 |
| AUC₀₋ₜₗₐₛₜ/Dose^{a} (min*kg*ng/ml/mg) | 2.96 | 1.31 |
| AUC_{0-inf} (min*ng/ml) | n/a | 1.44 |
| AUC_{0-inf}/Dose^{a} (min*kg*ng/ml/mg) | n/a | 1.30 |

The dose-normalised maximum plasma concentration (Cₘₐₓ/Dose) attained following administration of the THC-1 formulation was 7.08 kg*ng/ml/mg and was significantly higher (5.57-fold) than that following administration of the THC MCT tincture (1.27 kg*ng/ml/mg; P=0.0448) and 1.86-fold higher than that obtained following THC drops administration (3.81 ng/ml/μg; P=0.2916).

Administration of THC-1 formulation in a significantly higher (2.96-fold) dose-normalised AUC₀₋ₜₗₐₛₜ exposure (1440 min*kg*ng/ml/mg) when compared to THC MCT tincture administration (488 min*kg*ng/ml/mg, P=0.0078), but was comparable in exposure to commercial THC (1100 min*kg*ng/ml/mg, P=0.3698).

### Conclusion

The novel THC-1 formulation of the disclosure in which the THC was solubilized using α-cyclodextrin and poloxamer-407 has been shown to exhibit favourable PK parameters when administered to rats. The substantially faster tₘₐₓ denotes that this formulation provides a faster onset than both commercial THC and THC in MCT oil. In addition, the higher Cₘₐₓ demonstrated by the novel formulation provides the ability to administer lower doses to achieve the same effect in comparison to the other two formulations tested. This provides many advantages including a lower cost of goods and a lower side effect profile.

### Example 4: a Human Phase 1, 2-Part, Open-Label, Pilot Trial to Assess the Relative Bioavailability of Three Formulations of Cannabidiol (CBD) Under Fasted (Part A) and Fed (Part B) Conditions in Healthy Adult Male Subjects

A clinical study was undertaken to assess the relative bioavailability of two novel CBD formulations (T1 and T2) compared to a reference formulation (Epidiolex®) under fasting conditions in healthy adult male subjects.

As a second part of the study the effect of a high-fat, high-calorie meal on the bioavailability of the active ingredient CBD and its major metabolites 7-hydroxy-cannabidiol (7-OH-CBD) and 7-carboxy-cannabidiol (7-COOH-CBD), following a single dose of T1 or T2 in healthy adult male subjects.

### Study Design

A single-centre, Phase 1, randomized, open-label, 3-treatment, 4-period, 3-sequence crossover study was undertaken to compare the relative bioavailability of two novel CBD formulations (T1 and T2) and a reference formulation (Epidiolex®) under fasting conditions and to evaluate the effect of food on the bioavailability of T1 or T2 in healthy male subjects.

Part A consisted of three treatment periods. A total of 15 subjects were enrolled and randomized in one of three sequences, with a total of 5 subjects per sequence (n=5).

Subjects received a single dose of one of the three CBD formulations in each period, based on the randomization schedule. There was an at least 7-day (and no more than 14 days) washout between dosing.

Part A included a screening visit from Day-28 to Day-2. Eligible subjects were admitted to the clinical site on Day-1, with dosing occurring on Day 1 following at least 10 hours of fasting. Subjects were confined until the completion of the assessments on Day 3 and returned to the clinical site for outpatient follow-up (FU) visits on Days 4 and 5.

Subjects were re-admitted to the clinical site on Days 7 and 14 and received the second and third allocated treatment on Days 8 and 15, respectively. Subjects were confined until completion of the assessments on Days 10 and 17 for treatment periods 2 and 3, respectively and returned to the clinical site for FU visits on Days 11 and 12 in treatment Period 2 and Days 18 and 19 in treatment period 3.

Following at least 2 weeks washout period following last dose administration in Part A, subjects returned to the clinical site on Day 28 at earliest to continue in study Part B.

All subjects received a single dose of one test CBD formulation (T1), thirty minutes after the start of the high-fat, high-calorie breakfast. Subjects were confined until completion of the assessments on Day 31 and returned to the clinical site for follow up “FU” visits on Days 32 and 33.

### Study Treatments

In each treatment period the subjects received a single dose of one the following treatments:

### Part A:

Treatment A (Reference): 350 mg CBD (100 mg/mL CBD solution) Epidiolex® administered orally under fasting conditions.
Treatment B (T1): 350 mg CBD (87.5 mg capsules) administered orally under fasting conditions.
Treatment C (T2): 350 mg CBD (87.5 mg capsules) administered orally under fasting conditions.

### Part B:

Treatment D (T1): 350 mg CBD based on the PK and safety data from study Part A administered orally after a standard high-fat diet.

Details of the investigational products used are detailed in Table 16 below.

TABLE 16
Investigational product details

| | | | CBD Dose | |
|---|---|---|---|---|
| Investi- | | Route of | (mg) per | Inactive |
| gational | Dose | Adminis- | Adminis- | Ingredients/ |
| Product | Form | tration | tration | Excipients |
| Epidiolex ® | 100 | oral | 350 | Dehydrated alcohol, |
| | mg/ml | | | sesame seed oil, |
| | solution | | | strawberry flavour, |
| | | | | sucralose. |
| T1 formulation | capsules | oral | 350 | Hydroxypropyl-β- |
| | | | | cyclodextrin, |
| | | | | Poloxamer-407, |
| | | | | and Poloxamer-188 |
| T2 formulation | capsules | oral | 350 | Hydroxypropyl-β- |
| | | | | cyclodextrin, |
| | | | | Poloxamer-407, |
| | | | | Poloxamer-188, |
| | | | | Polysorbate 80, and |
| | | | | PEG 6000 |

### Preparation of Investigational Products:

The procedure for preparing the T1 and T2 formulations using the ingredients detailed in Tables 17 and 18 were as previously described.

TABLE 17
Formulation T1

| | | Composition | Quantity per |
|---|---|---|---|
| Ingredient | Source | (% w/w) | batch (grams) |
| Cannabidiol | Purisys | 22.6% | 98 |
| Poloxamer-407 | Spectrum | 11.1% | 48 |
| | Chemicals | | |
| Poloxamer-188 | Spectrum | 33.2% | 144 |
| | Chemicals | | |
| Hydroxypropyl-β- | Sigma-Aldrich | 33.2% | 144 |
| cyclodextrin | | | |

| | | |
|---|---|---|
| TOTAL | 100% | 434 |

TABLE 18
Formulation T2

| | | Composition | Quantity per |
|---|---|---|---|
| Ingredient | Source | (% w/w) | batch (grams) |
| Cannabidiol | Purisys | 17.1% | 98 |
| Poloxamer-407 | Spectrum | 4.2% | 24 |
| | Chemicals | | |
| Poloxamer-188 | Spectrum | 12.6% | 72 |
| | Chemicals | | |
| Hydroxypropyl-β- | Sigma-Aldrich | 33.6% | 192 |
| cyclodextrin | | | |
| Polysorbate 80 | Spectrum | 15.7% | 90 |
| | Chemicals | | |
| Polyethylene | Millipore Sigma | 16.8% | 96 |
| glycol 6000 | | | |

| | | |
|---|---|---|
| TOTAL | 100% | 572 |

### Blood Sample Collection for PK Analysis:

In each treatment period, a total of 19 blood samples were collected to analyse the blood for levels of CBD and its metabolites (7-OH-CBD and 7-COOH-CBD) plasma concentrations and PK analysis at the following time points: pre-dose, 5 min, 0.25, 0.5, 1, 1.5, 2, 2.5, 3, 4, 5, 6, 8, 12, 24-, 48-, 72- and 96-hours post-dose.

Total bile acids samples were collected in each treatment period pre-dose only. Levels of CBD, 7-OH-CBD and 7-COOH-CBD in plasma were analysed using validated ultra-performance liquid chromatography with tandem mass spectrometry bioanalytical methods for all subjects.

### Statistical Analyses

### PK Analysis:

Analysis of CBD, 7-OH-CBD and 7-COOH-CBD plasma concentration data are listed individually and summarized by each timepoint by treatment. Such data enable the ratios of CBD to the metabolites to be calculated.

PK parameters Cₘₐₓ, Tₘₐₓ, AUC₀₋ₜ, AUC_{0-∞} were determined using noncompartmental methods and summarized by treatment using descriptive statistics.

Comparison of exposure between treatments (the treatment ratio to support the geometric mean Frel of [Test 1] and [Test 2] compared with the Epidiolex® [Reference]) was investigated by performing linear fixed-effects model of log-transformed values of key exposure PK parameters AUC₀₋ₜ, AUC_{0-∞}, and Cₘₐₓ at the alpha level of 0.05, for each treatment.

Ratio of geometric least squares (LS) means, expressed as test/reference, and corresponding 90% confidence intervals (CI) are also presented.

Non-parametric analysis of the same comparisons was performed using a Wilcoxon signed-rank test for Tₘₐₓ. The median Tₘₐₓ for each treatment are presented along with the approximate 90% CI.

Additional modelling of untransformed Kₑₗ, and T_{1/2 el} were performed by ANOVA or non-parametric approaches. Ratio of geometric least squares (LS) means, expressed as test/reference, and corresponding 90% confidence intervals (CI) are presented, calculated for AUC₀₋ₜ, AUC_{0-∞}, and Cₘₐₓ). Also, Cₘₐₓ, AUC and Frel of the sum of CBD and 7-OH-CBD, were calculated (where possible) and using the single dose data, extrapolation to steady state at 12 h dose interval using WinNonLin or similar were modelled.

### Standards for Biosimilarity Acceptance:

The 90% CIs for the ratio of geometric means (Test formulation/Reference formulation) based on least squares means from the ANOVA of the In-transformed AUC_{0-∞}, must be within 80.00% to 125.00%.

### Safety and Tolerability Analysis:

Safety and tolerability of Epidiolex®, T1 and T2 formulations were evaluated through the assessment of AEs (i.e., seriousness, severity, relationship to the study drug, outcome, duration, and management), vital signs, 12-lead ECGs, clinical laboratory tests, and physical examinations.

Medical history and AE terms were coded using the Medical Dictionary for Regulatory Activities (MedDRA). Prior and concomitant medications were coded using World Health Organization-Drug Dictionary (WHO-DD).

Individual AEs, clinical laboratory data along with indication if outside the normal reference range; and physical examination findings (including changes from baseline) were listed. Additionally clinical laboratory data, VS and ECGs parameters are presented descriptively (where applicable).

### Results

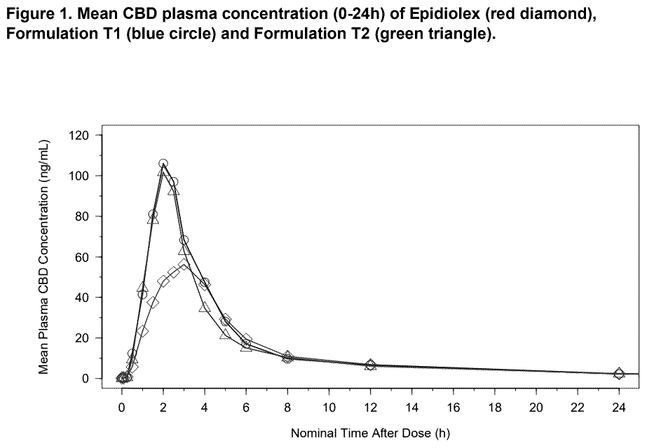

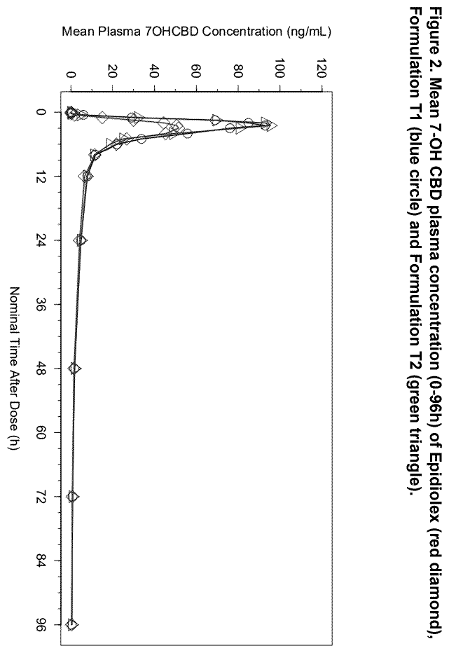

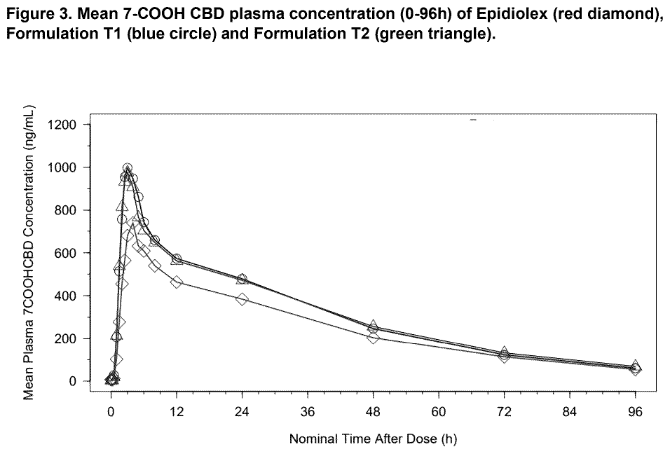

The mean plasma concentrations of CBD, 7-OH-CBD and 7-COOH-CBD for Epidiolex® and the two different CBD formulations after dosing in Part A (fasted conditions) are detailed by each timepoint in Tables 19A-C. FIGS. 1 to 3 detail the mean plasma concentrations of CBD, 7-OH CBD and 7-COOH CBD during the study.

TABLE 19A
Mean plasma concentration of CBD, 7-OH-CBD and 7-COOH-CBD
after dosing with Epidiolex (RTM) in Part A (Fasted)

| Time | CBD | 7-OH CBD | 7-COOH CBD |
|---|---|---|---|
| (h) | (ng/ml) | (ng/ml) | (ng/ml) |
| 0 | 0.2341 | 0.1056 | 7.273 |
| 0.083 | 0.2388 | 0.07534 | 6.486 |
| 0.25 | 0.8830 | 0.1845 | 6.950 |
| 0.5 | 5.681 | 1.1740 | 11.82 |
| 1.0 | 23.30 | 15.00 | 102.5 |
| 1.5 | 37.45 | 30.05 | 277.5 |
| 2.0 | 47.93 | 44.35 | 455.1 |
| 2.5 | 52.39 | 50.59 | 564.2 |
| 3.0 | 56.28 | 51.45 | 679.5 |
| 4.0 | 46.21 | 45.27 | 739.1 |
| 5.0 | 29.21 | 26.81 | 631.2 |
| 6.0 | 19.36 | 21.83 | 608.1 |
| 8.0 | 10.93 | 11.41 | 539.1 |
| 12.0 | 6.837 | 6.471 | 463.1 |
| 24.0 | 2.404 | 4.114 | 383.8 |
| 48.0 | 1.229 | 1.464 | 203.4 |
| 72.0 | 0.7079 | 0.6239 | 113.2 |
| 96.0 | 0.5666 | 0.3663 | 54.01 |

TABLE 19B
Mean plasma concentration of CBD, 7-OH-CBD and 7-COOH-
CBD after dosing with Formulation T1 in Part A (Fasted)

| Time | CBD | 7-OH CBD | 7-COOH CBD |
|---|---|---|---|
| (h) | (ng/ml) | (ng/ml) | (ng/ml) |
| 0 | 0.2040 | 0.06571 | 5.531 |
| 0.083 | 0.1982 | 0.05721 | 5.147 |
| 0.25 | 0.4462 | 0.08291 | 5.380 |
| 0.5 | 12.35 | 5.935 | 27.74 |
| 1.0 | 41.36 | 29.16 | 206.7 |
| 1.5 | 81.06 | 68.86 | 513.4 |
| 2.0 | 106.0 | 84.95 | 756.5 |
| 2.5 | 97.04 | 92.82 | 954.8 |
| 3.0 | 68.39 | 75.94 | 997.0 |
| 4.0 | 47.42 | 55.79 | 947.3 |
| 5.0 | 28.01 | 33.79 | 861.8 |
| 6.0 | 17.04 | 22.19 | 742.3 |
| 8.0 | 9.658 | 11.65 | 659.1 |
| 12.0 | 6.576 | 7.829 | 572.9 |
| 24.0 | 2.172 | 4.788 | 478.3 |
| 48.0 | 10.72 | 1.551 | 246.1 |
| 72.0 | 0.7379 | 0.7770 | 122.6 |
| 96.0 | 0.5263 | 0.3466 | 60.08 |

TABLE 19C
Mean plasma concentration of CBD, 7-OH-CBD and 7-COOH-
CBD after dosing with Formulation T2 in Part A (Fasted)

| Time | CBD | 7-OH CBD | 7-COOH CBD |
|---|---|---|---|
| (h) | (ng/ml) | (ng/ml) | (ng/ml) |
| 0 | 0.1536 | 0.03789 | 3.224 |
| 0.083 | 0.1532 | 0.03945 | 3.162 |
| 0.25 | 0.5725 | 0.2004 | 3.344 |
| 0.5 | 9.333 | 4.563 | 18.33 |
| 1.0 | 44.51 | 32.10 | 213.5 |
| 1.5 | 78.15 | 69.40 | 537.6 |
| 2.0 | 101.7 | 92.80 | 815.1 |
| 2.5 | 92.26 | 95.46 | 932.2 |
| 3.0 | 62.81 | 80.55 | 975.0 |
| 4.0 | 34.59 | 48.89 | 907.8 |
| 5.0 | 21.22 | 25.05 | 765.4 |
| 6.0 | 15.02 | 18.68 | 703.4 |
| 8.0 | 10.29 | 11.05 | 647.3 |
| 12.0 | 6.041 | 7.542 | 562.0 |
| 24.0 | 2.329 | 4.699 | 471.2 |
| 48.0 | 1.172 | 1.802 | 256.0 |
| 72.0 | 0.6404 | 0.7906 | 131.9 |
| 96.0 | 0.4939 | 0.4089 | 68.46 |

The mean PK parameters T_{lag}, Tₘₐₓ, Cₘₐₓ, AUC₀₋ₗₐₛₜ, AUC_{0-∞}, Kel, t_{1/2}, CI/F and Vz/F after dosing in Part A (fasted conditions) for Epidiolex® and the two different CBD formulations detailed Table 19D.

TABLE 19D
Mean PK parameters of Epidiolex (RTM) and
Formulation T1 and T2 in Part A (Fasted)

| | T_{lag} | Tₘₐₓ | Cₘₐₓ | AUC₀₋ₗₐₛₜ | AUC_{0-inf} | Kel | t½ | CI/F | Vz/F |
|---|---|---|---|---|---|---|---|---|---|
| Formulation | (h) | (h) | (ng/ml) | (h*ng/ml) | (h*ng/ml) | (h⁻¹) | (h) | (L/h) | (L) |
| Epidiolex | 0.065 | 3.07 | 71.78 | 400.7 | 422.8 | 0.022 | 34.30 | 829.7 | 40510 |
| T1 | 0.030 | 2.18 | 123.7 | 474.0 | 518.1 | 0.022 | 36.03 | 659.3 | 34390 |
| T2 | 0.054 | 2.18 | 116.9 | 444.9 | 469.3 | 0.023 | 33.95 | 786.3 | 37170 |

As can be seen, under Part A, fasted conditions, both T1 and T2 formulations were seen to produce higher Cₘₐₓ and AUC than Epidiolex®.

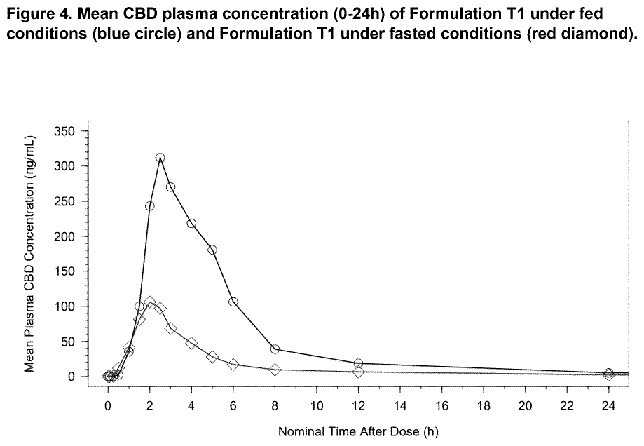

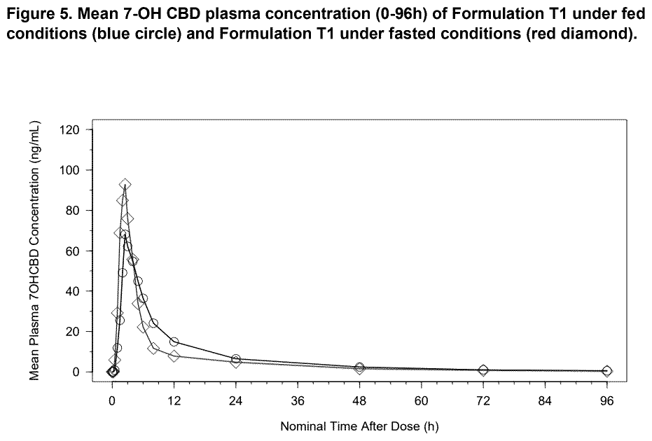

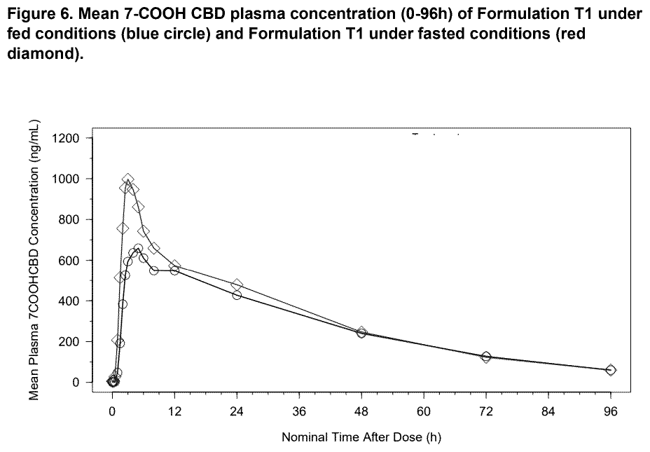

Following Part A of the study, formulation T1 was chosen for dosing under Part B whereby the formulation was dosed 30 minutes after a high-fat meal. The mean plasma concentrations of CBD, 7-OH-CBD and 7-COOH-CBD for the T1 formulation after dosing under fed conditions are detailed by each timepoint in Table 20A. FIGS. 4 to 6 detail the mean plasma concentrations of CBD, 7-OH CBD and 7-COOH CBD respectively which were recorded for formulation T1 after fed and fasted conditions.

TABLE 20A
Mean plasma concentration of CBD, 7-OH-CBD and 7-COOH-
CBD after dosing with Formulation T1 in Part B (Fed)

| Time | CBD | 7-OH CBD | 7-COOH CBD |
|---|---|---|---|
| (h) | (ng/ml) | (ng/ml) | (ng/ml) |
| 0 | 0.06692 | 0.004308 | 0.5745 |
| 0.083 | 0.06705 | 0.004162 | 0.5474 |
| 0.25 | 0.1622 | 0.01400 | 0.5050 |
| 0.5 | 2.113 | 0.8684 | 2.191 |
| 1.0 | 35.36 | 11.87 | 47.59 |
| 1.5 | 99.87 | 25.53 | 190.9 |
| 2.0 | 243.1 | 49.05 | 383.1 |
| 2.5 | 311.9 | 68.19 | 525.2 |
| 3.0 | 270.1 | 62.19 | 593.9 |
| 4.0 | 218.5 | 54.79 | 636.1 |
| 5.0 | 180.8 | 44.90 | 658.8 |
| 6.0 | 106.3 | 36.35 | 610.6 |
| 8.0 | 38.85 | 24.09 | 548.3 |
| 12.0 | 18.68 | 14.91 | 547.7 |
| 24.0 | 5.153 | 6.508 | 426.8 |
| 48.0 | 2.799 | 2.433 | 239.5 |
| 72.0 | 1.814 | 1.020 | 127.1 |
| 96.0 | 2.128 | 0.6146 | 59.58 |

The mean PK parameters T_{lag}, Tₘₐₓ, Cₘₐₓ, AUC₀₋ₗₐₛₜ, AUC_{0-∞}, Kel, t₁₋₂, CI/F and Vz/F after dosing in Part A (fasted conditions) for Epidiolex® and the two different CBD formulations detailed Table 20B.

TABLE 20B
Mean PK parameters of Formulation T1 in Part B (Fed)

| | T_{lag} | Tₘₐₓ | Cₘₐₓ | AUC₀₋ₗₐₛₜ | AUC_{0-inf} | Kel | t½ | CI/F | Vz/F |
|---|---|---|---|---|---|---|---|---|---|
| Formulation | (h) | (h) | (ng/ml) | (h*ng/ml) | (h*ng/ml) | (h⁻¹) | (h) | (L/h) | (L) |
| T1 | 0.026 | 3.11 | 395.2 | 1549 | 1547 | 0.018 | 41.30 | 241.3 | 14330 |

As can be seen, under Part B, fed conditions the T1 CBD formulation produced much higher Cₘₐₓ and AUC than under fasted conditions.

### Conclusion

The use of the novel CBD formulations of the disclosure produced a more favourable plasma concentration and PK parameters than those of Epidiolex® and as such these formulations are suitable for development for commercial use.

### Example 5: Study on Various Combinations of Excipients

In order to determine the various excipients and their ratios, different CBD-containing formulations were prepared using the methodology as previously detailed. The combinations of excipients were as detailed in Table 21 below.

TABLE 21
Combinations of excipients used.

| | CBD | Excipient 1 | Excipient 2 | Excipient 3 | Excipient 4 | Excipient 5 |
|---|---|---|---|---|---|---|
| ID | (mg) | (mg) | (mg) | (mg) | (mg) | (mg) |
| 8A | 2 | Poloxamer- | Alpha-CD | — | — | — |
| | | 407 (8) | (2) | | | |
| SP-1 | 2 | HP-b-CD | — | — | — | — |
| | | (14) | | | | |
| SP-2 | 2 | HP-b-CD | — | — | — | — |
| | | (8) | | | | |
| SP-3 | 2 | Poloxamer- | HP-b-CD | — | — | — |
| | | 407 (0.5) | (7) | | | |
| SP-4 | 2 | Poloxamer- | Poloxamer- | HP-b-CD | — | — |
| | | 407 (0.5) | 188 (1.5) | (6) | | |
| SP-5 | 2 | Poloxamer- | Poloxamer- | HP-b-CD | PEG-3350 | — |
| | | 407 (0.5) | 188 (1.5) | (3) | (3) | |
| SP-6 | 2 | HP-b-CD | Gelucire | — | — | — |
| | | (3) | 48/16 (7) | | | |
| SP-7 | 2 | HP-b-CD | Gelucire | — | — | — |
| | | (3) | 50/13 (7) | | | |
| SP-8 | 2 | HP-b-CD | Gelucire | — | — | — |
| | | (3) | 59/14 (7) | | | |
| SP-9 | 2 | Poloxamer- | Poloxamer- | HP-b-CD | Gelucire | |
| | | 407 (0.5) | 188 (1.5) | (2) | 48/16 (3) | |
| SP-10 | 2 | Poloxamer- | Poloxamer- | Alpha-CD | Gelucire | — |
| | | 407 (0.5) | 188 (1.5) | (2) | 48/16 (3) | |
| SP-11 | 2 | Poloxamer- | Poloxamer- | HP-b-CD | Gelucire | — |
| | | 407 (0.5) | 188 (1.5) | (2) | 59/14 (3) | |
| SP-12 | 2 | Poloxamer- | Poloxamer- | HP-b-CD | Gelucire | PEG-1500 |
| | | 407 (0.5) | 188 (1.5) | (2) | 59/14 (1) | (2) |
| SP-13 | 2 | Poloxamer- | Poloxamer- | HP-b-CD | Gelucire | PEG-1500 |
| | | 407 (0.5) | 188 (1.5) | (2) | 59/14 (2) | (1) |
| SP-14 | 2 | Poloxamer- | Poloxamer- | HP-b-CD | — | — |
| | | 407 (1) | 188 (3) | (3) | | |
| SP-15 | 2 | Poloxamer- | Poloxamer- | HP-b-CD | — | — |
| | | 407 (2) | 188 (6) | (2) | | |
| SP-16 | 2 | Poloxamer- | Poloxamer- | HP-b-CD | Tween 20 | PEG-1500 |
| | | 407 (0.5) | 188 (1.5) | (3) | (2) | (1) |
| SP-14-B | 2 | Poloxamer- | Poloxamer- | HP-b-CD | — | — |
| | | 407 (1) | 188 (3) | (3) | | |
| SP-17 | 2 | Poloxamer- | HP-b-CD | — | — | — |
| | | 407 (8) | (2) | | | |
| SP-18 | 2 | Poloxamer- | Poloxamer- | HP-b-CD | Polysorbate | PEG-1500 |
| | | 407 (0.5) | 188 (1.5) | (3) | 80 (2) | (1) |
| SP-19 | 2 | Poloxamer- | Poloxamer- | HP-b-CD | Polysorbate | PEG-1500 |
| | | 407 (0.5) | 188 (1.5) | (4) | 80 (1) | (1) |
| SP-20 | 2 | Poloxamer- | Poloxamer- | HP-b-CD | Polysorbate | — |
| | | 407 (0.5) | 188 (1.5) | (6) | 80 (1) | |
| SP-21 | 2 | Poloxamer- | Poloxamer- | HP-b-CD | Polysorbate | PEG-3350 |
| | | 407 (0.5) | 188 (1.5) | (4) | 80 (2) | (1) |
| SP-22 | 2 | Poloxamer- | Poloxamer- | HP-b-CD | Polysorbate | PEG-3350 |
| | | 407 (0.5) | 188 (1.5) | (4) | 80 (1.4) | (1) |
| SP-23 | 2 | Poloxamer- | Poloxamer- | HP-b-CD | Polysorbate | Sorbitol (2) |
| | | 407 (0.5) | 188 (1.5) | (4) | 80 (2) | |
| SP-24 | 2 | Poloxamer- | Poloxamer- | HP-b-CD | Polysorbate | PEG-6000 |
| | | 407 (0.5) | 188 (1.5) | (4) | 80 (2) | (2) |
| SP-25 | 2 | Poloxamer- | Poloxamer- | HP-b-CD | Polysorbate | PEG-1500 |
| | | 407 (0.5) | 188 (1.5) | (4) | 80 (2) | (2) |
| SP-26 | 2 | Poloxamer- | Poloxamer- | HP-b-CD | Polysorbate | PEG-6000 |
| | | 407 (0.5) | 188 (1.5) | (4) | 60 (2) | (2) |

As can be seen above CBD was successfully formulated using a combination of one or more cyclodextrins or cyclodextrin derivatives, and one or more poloxamers and optionally one or more pharmaceutically acceptable emulsifiers and/or surfactants.

The formulations were tested in a dissolution assay to assess their disintegration times and dissolution characteristics.

All formulations had suitable dissolution characteristics.

### Example 6: Stability Study

The capsules prepared for the human Phase I study were tested to determine their stability profile and ensure they were suitable for use.

Tables 22 to 25 detail the stability data obtained on the two formulations.

TABLE 22
Stability data of Formulation T1 at 25° C. / 60% RH

| Test | Specification | T = 0 | T = 1M |
|---|---|---|---|
| Appearance | Size 0 white opaque | Conforms | Conforms |
| | coni-snap capsules | | |
| CBD assay | 90-110% of label claim | 99.78% | 100.6% |
| | (87.5 mg) | | |
| Delta-9-THC | Report results | ND | ND |
| Delta-8-THC | Report results | ND | ND |
| Individual | Report results | ND | ND |
| unspecified | | | |
| impurities | | | |
| Total impurities | Report results | ND | ND |
| Water content | Report results | 0.000% | 0.000% |
| Total aerobic | NMT 10³ CFU/g | Pass | Pass |
| microbial count | | | |
| Total combined | NMT 102 CFU/g | Pass | Pass |
| yeasts/mold count | | | |
| E. coli | Absent/1 g | Pass | Pass |

TABLE 23
Stability data of Formulation T1 at 40° C. / 75% RH

| Test | Specification | T = 0 | T = 1M |
|---|---|---|---|
| Appearance | Size O white opaque | Conforms | Conforms |
| | coni-snap capsules | | |
| CBD assay | 90-110% of label claim | 99.78% | 98.19% |
| | (87.5 mg) | | |
| Delta-9-THC | Report results | ND | ND |
| Delta-8-THC | Report results | ND | ND |
| Individual | Report results | ND | ND |
| unspecified | | | |
| impurities | | | |
| Total impurities | Report results | ND | ND |
| Water content | Report results | 0.000% | 0.000% |
| Total aerobic | NMT 10³ CFU/g | Pass | Pass |
| microbial count | | | |
| Total combined | NMT 102 CFU/g | Pass | Pass |
| yeasts/mold count | | | |
| E. coli | Absent/1 g | Pass | Pass |

TABLE 24
Stability data of Formulation T2 at 25° C. / 60% RH

| Test | Specification | T = 0 | T = 1M |
|---|---|---|---|
| Appearance | Size 0 white opaque | Conforms | Conforms |
| | coni-snap capsules | | |
| CBD assay | 90-110% of label claim | 101.8% | 100.3% |
| | (87.5 mg) | | |
| Delta-9-THC | Report results | ND | ND |
| Delta-8-THC | Report results | ND | ND |
| Individual | Report results | ND | ND |
| unspecified | | | |
| impurities | | | |
| Total impurities | Report results | ND | ND |
| Water content | Report results | 0.000% | 0.000% |
| Total aerobic | NMT 10³ CFU/g | Pass | Pass |
| microbial count | | | |
| Total combined | NMT 102 CFU/g | Pass | Pass |
| yeasts/mold count | | | |
| E. coli | Absent/1 g | Pass | Pass |

TABLE 25
Stability data of Formulation T2 at 40° C. / 75% RH

| Test | Specification | T = 0 | T = 1M |
|---|---|---|---|
| Appearance | Size 0 white opaque | Conforms | Conforms |
| | coni-snap capsules | | |
| CBD assay | 90-110% of label claim | 99.78% | 97.85% |
| | (87.5 mg) | | |
| Delta-9-THC | Report results | ND | ND |
| Delta-8-THC | Report results | ND | ND |
| Individual | Report results | ND | ND |
| unspecified | | | |
| impurities | | | |
| Total impurities | Report results | ND | ND |
| Water content | Report results | 0.000% | 0.000% |
| Total aerobic | NMT 10³ CFU/g | Pass | Pass |
| microbial count | | | |
| Total combined | NMT 102 CFU/g | Pass | Pass |
| yeasts/mold count | | | |
| E. coli | Absent/1 g | Pass | Pass |

As can be seen the two formulations were deemed stable at both normal and accelerated stability.

### Example 7: Enhanced Drug Loading of CBD Formulation

CBD is a hydrophobic molecule which is virtually insoluble in aqueous systems. Once ingested CBD is primarily absorbed through passive diffusion in the gastrointestinal tract. Various different methods have been employed by scientists wishing to enhance the solubility of CBD formulations including use of lipid formulations, encapsulation of the CBD to form micelles, and the preparation of nanoemulsions of CBD.

These technologies result in low drug loading and poor stability. They are complex processes with a high cost.

The aim of the current example was to enhance the drug loading of CBD in the present formulations.

The formulation used in the present example is that of SP-14, which comprises CBD at approximately 22% (w/w), poloxamer-407 at approximately 11% (w/w), poloxamer-188 at approximately 33% (w/w) and hydroxypropyl-beta-cyclodextrin at approximately 33% (w/w).

However, the skilled person would appreciate that any of the exemplified formulations of the disclosure would be suitable to be used for the improved filling technique to achieve enhanced drug loading of cannabinoid.

### Methods

Formulation SP-14 from Table 21 was prepared as follows:

The ingredients were weighed using a calibrated balance and transferred to a clean and dry wide mouth glass jar.

The glass jar was closed with a SureTight lid and shaken well for 2-3 minutes to mix and seal it with parafilm.

The powder mix was prepared in a 150 ml stainless-steel high-pressure reactor. All ingredients were added, and the reactor was sealed prior to the addition of the solvent. CO₂ was added and pressure and temperature were increased to reach the supercritical phase. Reactor was held between 2000 and 3000 psi with a temperature between 3° and 50° C. and the contents were vigorously mixed for between 20 and 60 minutes. The reactor was depressurized over 1 hour and the resultant inclusion complex powder was un-flowable and tacky in consistency. This material was collected into an airtight and waterproof container and placed in a water bath heated to 50° C.

Once warmed the material became semi-liquid with the flow characteristics of toothpaste. This material was transferred into a syringe whilst still warm.

The material was dispensed via the syringe into the larger side of a two-piece size 0 capsule and the two pieces of the capsule joined.

Capsules were then stored at room temperature in the dark.

Analysis of the quantity of CBD was then undertaken to determine the drug loading of the capsule using this enhanced filling method.

### Results

Analysis of the filled capsule resulted in a fill of 680 mg of formulation per size 0 capsule.

This equates to a drug loading of 150 mg (22%) CBD.

In the case of a size 00 capsule a 15% higher loading of CBD could be achieved resulting in 172.5 mg of CBD per capsule. Smaller capsules can also be filled resulting in a 15% lower CBD loading of 127.5 mg of CBD per capsule.

### Conclusion

The enhanced drug loading of 150 mg of CBD per capsule is very significant. Such a high drug loading has not heretofore been achieved in the literature for a solid oral dosage form.

The resultant capsule enables the capsule burden for patients to be lessened. For example, the approved dose of CBD in Epidiolex is 20-25 mg/kg/day. In a 70 kg adult this equates to 1,400-1,750 mg of CBD per day.

Standard filling techniques usually produce capsules comprising a maximum of 80 mg of CBD, but more commonly around 50 mg of CBD. The patient would need to take between 17 to 28 capsules per day.

The use of the higher drug loading CBD formulation exemplified in this example would result in a far lower capsule burden for the patient of around 9 to 11 capsules per day.

The filling methodology is cost effective and scalable and the reduction in capsule burden ensures an enhanced patient experience.

### Example 8: Maximising Deliverable Dose of CBD

Example 7 above describes the use of an innovative filling methodology whereby the capsules can be loaded with a larger quantity of formulation resulting in a higher drug loading of CBD and a reduced capsule burden to the patient.

The present example details experiments undertaken whereby the amounts of the various excipients in formulation SP-14 were altered in order to determine whether a higher concentration of CBD could be loaded into the capsule, thereby increasing the deliverable dose of CBD per capsule.

### Methods

Excipients poloxamer-407 (p407), poloxamer-188 (P188) and hydroxypropyl-beta-cyclodextrin (HP-b-CD) and active cannabidiol (CBD) were mixed in the percentages detailed in Table 26 below.

Formulations were prepared and loaded into a size 0 HPMC capsule as detailed in Example 7.

Capsules were accurately weighed and the amount of API and excipients loaded were recorded. The potency of the CBD was measured in ppm and converted into percentage and recorded.

Dissolution testing of formulations AVT-121 to AVT-128 was then undertaken using industry standard dissolution testing methodologies.

The heat stability of formulations AVT-121 to AVT-124 were tested using various processing temperatures to determine whether CBD was lost through the capsule loading process.

TABLE 26
Percentage of CBD and excipients.

| | CBD | p407 | p188 | HP-β-CD |
|---|---|---|---|---|
| Code | (%) | (%) | (%) | (%) |
| AVT-103 | 41.7 | 8.3 | 25.0 | 25.0 |
| AVT-104 | 46.2 | 7.7 | 23.1 | 23.1 |
| AVT-107 | 35.3 | 11.7 | 35.3 | 17.7 |
| AVT-121 | 33.1 | 11.2 | 33.4 | 22.3 |
| AVT-122 | 33.1 | 8.0 | 38.0 | 20.9 |
| AVT-123 | 33.1 | 8.0 | 28.9 | 30.0 |
| AVT-124 | 33.1 | 10.0 | 30.0 | 26.9 |
| AVT-125 | 33.1 | 10.5 | 34.0 | 22.4 |
| AVT-126 | 33.1 | 10.5 | 22.9 | 33.5 |
| AVT-127 | 34.5 | 10.5 | 32.6 | 22.4 |
| AVT-128 | 34.5 | 10.5 | 22.9 | 32.1 |
| SP-14 | 22.2 | 11.1 | 33.3 | 33.3 |

### Results

Table 27 below details the measured potency of the CBD in the various different formulations produced. All formulations produced a higher potency of CBD compared to the baseline formulation SP-14.

The amount of formulation that was able to be filled into the size 0 capsule all increased from 675 mg baseline (SP-14 formulation). The amounts that were able to be filled in a size 0 capsule were from 725 mg to 805 mg with the new formulations.

The amount of CBD per capsule also increased with all new formulations. For the baseline formulation (SP-14) the amount of CBD able to be filled into a capsule was 152 mg.

The improved formulations provided between 230 mg of CBD per capsule to 310 mg of CBD per capsule, a substantial increase in the amount of CBD per unit dose.

TABLE 27
Potency, capsule fill and amount of CBD per capsule.

| | Measured Potency | Capsule filling | CBD per |
|---|---|---|---|
| | of CBD | amount | capsule |
| Code | (%) | (mg) | (mg) |
| AVT-103 | 35.6 | 725 | 258.0 |
| AVT-104 | 42.7 | 725 | 310.0 |
| AVT-107 | 33.8 | 725 | 245.0 |
| AVT-121 | 30.4 | 759 | 230.5 |
| AVT-122 | 33.0 | 775 | 255.6 |
| AVT-123 | 32.3 | 783 | 252.9 |
| AVT-124 | 32.1 | 759 | 243.4 |
| AVT-125 | 32.2 | 794 | 255.4 |
| AVT-126 | 33.4 | 805 | 268.8 |
| AVT-127 | 33.4 | 795 | 265.8 |
| AVT-128 | 33.0 | 791 | 261.3 |
| SP-14 | 22.5% | 675 | 152.0 |

The dissolution characteristics of formulations AVT-121 to AVT-128 were tested and results are detailed in Table 28 below.

TABLE 28
Dissolution characteristics of formulations.

| | CBD recovered | Average CBD recovered |
|---|---|---|
| Code | (mg) | (%) |
| AVT-121 | 207.8 | 90.1 |
| AVT-122 | 191.0 | 74.7 |
| AVT-123 | 134.8 | 53.4 |
| AVT-124 | 154.9 | 63.6 |
| AVT-125 | 215.7 | 84.5 |
| AVT-126 | 172.2 | 63.8 |
| AVT-127 | 200.0 | 75.3 |
| AVT-128 | 164.0 | 62.6 |

The heat stability of CBD through the capsule loading process of formulations AVT-121 to AVT-124 are detailed in Table 29 below.

TABLE 29
Heat stability of CBD through capsule loading process.

| | Process | | Average | CBD |
|---|---|---|---|---|
| | temperature | Time | potency | remaining |
| Code | (° C.) | (min) | (ppm) | (%) |
| AVT-121 | n/a | Initial | 317363.3 | 100.0 |
| | 40 | 10 | 316689.7 | 99.8 |
| | 40 | 20 | 314968.4 | 99.2 |
| | 40 | 30 | 308256.7 | 97.1 |
| | 50 | 10 | 315539.0 | 99.4 |
| | 50 | 20 | 317985.8 | 100.2 |
| | 50 | 30 | 317755.6 | 100.1 |
| AVT-122 | n/a | Initial | 329981.1 | 100.0 |
| | 50 | 30 | 316916.7 | 96.0 |
| AVT-123 | n/a | Initial | 322842.3 | 100.0 |
| | 50 | 30 | 309537.6 | 95.9 |
| AVT-124 | n/a | Initial | 320666.7 | 100.0 |
| | 50 | 30 | 318670.9 | 99.4 |

### Conclusion

The present example demonstrates that by altering the percentage of the excipients in the formulation and using the novel injection capsule filling technique the amount of CBD per capsule can be maximised substantially. This presents clear advantages by enabling a higher amount of active ingredient to be delivered in fewer unit doses.

The dissolution characteristics of the formulations AVT-121 to AVT-128 demonstrate improved properties with a high percentage of CBD recovered in almost all cases.

Stability testing of the injection capsule filling method detailed in Example 7 with the formulations of the present example show excellent stability at all experimentally tested conditions.

### Overall Conclusion and Summary

The application details the various challenges associated with the development of a cannabinoid formulation and how the inventors have overcome these challenges.

The first challenge was the preparation of a solid dosage form of cannabinoids, in the case illustrated, CBD. At the present time CBD is only available to be prescribed as Epidiolex® which is formulated in sesame oil.

This results in adults that are by nature heavier than children needing to take large quantities of sesame oil per day which has gastrointestinal side effects. In addition, children and adults that have a sesame allergy would be unable to be prescribed Epidiolex®.

The examples above demonstrate that cannabinoids can be formulated into a solid dosage form. This dosage form has been shown to be stable and not produce side effects in a Phase 1 clinical trial. The data from the clinical trial also showed that the bioavailability of the CBD was commensurate with the reference compound Epidiolex®.

The second challenge faced by the inventors was how to increase the amount of cannabinoid that can be incorporated into a single dosage form. Until now solid dosage forms of CBD were unsuitable for use due to the small amount of CBD that was able to be encapsulated in a dosage form. Due to the relatively high dose of CBD that is required to be efficacious formulations comprising a small amount of CBD means that a patient would need to take a large number of pills per day.

As the above examples demonstrate, the use of a specific excipients in combination with a specific processing methodology has enabled a high drug loading of CBD to be achieved per unit.

The final challenge presented by the preparation of solid dose formulations of cannabinoids was the regulatory challenge presented by the excipients which all have a maximum daily allowance defined by the FDA. For example, the FDA details the maximum daily amount of poloxamer-407 to be 495 mg; poloxamer-188 to be 1,800 mg; and HP-β-CD to be 8,000 mg.

When considering the content of these excipients in a unit dose there is a challenge to ensure the amounts of excipients will fall under the limits, whilst also being able to deliver an efficacious dose of CBD.

As can be seen in the examples above and Table 30 below, the inventors have achieved a solid dose form of CBD with excipients which fall within the limits set by the FDA for the excipients.

TABLE 30
Amount of cannabinoid / excipient per unit dose form.

| | CBD | Poloxamer-407 | Poloxamer-188 | HP-β-CD |
|---|---|---|---|---|
| Code | (mg) | (mg) | (mg) | (mg) |
| AVT-103 | 302.33 | 60.18 | 181.25 | 181.25 |
| AVT-104 | 334.95 | 55.83 | 167.48 | 167.48 |
| AVT-107 | 255.93 | 84.83 | 255.93 | 128.33 |
| AVT-121 | 251.23 | 85.01 | 253.51 | 169.26 |
| AVT-122 | 256.53 | 62.00 | 294.50 | 161.98 |
| AVT-123 | 259.17 | 62.64 | 226.29 | 234.90 |
| AVT-124 | 251.23 | 75.90 | 227.70 | 204.17 |
| AVT-125 | 262.81 | 83.37 | 269.96 | 177.86 |
| AVT-126 | 266.46 | 84.53 | 184.35 | 269.68 |
| AVT-127 | 274.28 | 83.48 | 259.17 | 178.08 |
| AVT-128 | 272.90 | 83.06 | 181.14 | 253.91 |
| SP-14 | 149.85 | 74.93 | 224.78 | 224.78 |

1. A solid, oral pharmaceutical dosage form comprising cannabidiol, hydroxypropyl-β-cyclodextrin (HPBCD), poloxamer-188, and poloxamer-407, wherein the cannabidiol is present in an amount from 100-300 mg per unit dose.

2. A solid, oral pharmaceutical dosage form according to claim 1, wherein the cannabidiol is present in an amount of between 20% (w/w) to 50% (w/w) of the total composition.

3. A solid, oral pharmaceutical dosage form according to claim 1, wherein the hydroxypropyl-β-cyclodextrin (HPBCD) is present in an amount of between 15% (w/w) and 35% (w/w).

4. A solid, oral pharmaceutical dosage form according to claim 1, wherein the poloxamer-188 is present in an amount of between 20% (w/w) to 40% (w/w).

5. A solid, oral pharmaceutical dosage form according to claim 1, wherein the poloxamer-407 is present in an amount of between 5% (w/w) to 15% (w/w).

6. A solid, oral pharmaceutical dosage form according to claim 1, wherein the formulation further comprises one or more pharmaceutically acceptable emulsifier and/or surfactant.

7. A solid, oral pharmaceutical dosage form according to claim 6, wherein the pharmaceutically acceptable emulsifier and/or surfactant is selected from the group consisting of: lecithin, glyceryl monostearate, methylcellulose, sodium lauryl sulfate, sodium oleate, sorbitan monopalmitate, sorbitan monostearate, sorbitan tristearate, tragacanth, triethanolamine oleate, polyethylene sorbitan monolaurate, polyethylene glycol (PEG), macrogol 3350, macrogol 4000, macrogol 6000, detergents, polysorbate 80 (polyoxyethylene sorbitan monooleate), polysorbate 20 (polyoxyethylene sorbitan monolaurate), cetearyl glucoside, polyglucosides, sorbitan monooleate (Span® 80), sorbitan monolaurate (Span® 20), polyoxyethylene monostearate (Myrj 45), polyoxyethylene vegetable oil (Emulphor®), cetyl piridinium chloride, polysaccharides gums, xanthan gums, tragacanth, gum arabica, and acacia.

8. A solid, oral pharmaceutical dosage form according to claim 6, wherein the pharmaceutically acceptable emulsifier and/or surfactant is present in an amount of between 0.1% (w/w) to 35% (w/w) of the total composition.

9. A solid, oral pharmaceutical dosage form according to claim 1, wherein the dosage form is formulated for administration as a dosage form selected from the group consisting of: tablet, pill, pellet, capsule, powder, lozenge, granule, and sustained-release formulation.

10. A solid, oral pharmaceutical dosage form according to claim 1, wherein the dosage form is formulated for administration before food, with food or after food.

11. A solid, oral pharmaceutical dosage form according to claim 1, wherein the cannabidiol is present in an amount of 250 mg per unit dose.

12. A solid, oral pharmaceutical dosage form according to claim 1, wherein the dosage form produces an AUC₀₋ₜ which is between 80% and 125% of the AUC₀₋ₜ of a reference compound Epidiolex® following administration of the dosage form to a subject.

13. A solid, oral pharmaceutical dosage form according to claim 1, wherein the dosage form produces a Cₘₐₓ which is between 80% and 125% of the Cₘₐₓ of a reference compound Epidiolex® following administration of the dosage form to a subject.

14. A method of treatment comprising administering to a subject a therapeutically effective amount of the solid oral dosage form according to claim 1.

15. A solid, oral pharmaceutical dosage form comprising per unit dose cannabidiol in an amount of from 100-300 mg, hydroxypropyl-β-cyclodextrin (HPβCD) in an amount of between 15% (w/w) and 35% (w/w), poloxamer-188 in an amount of between 20% (w/w) to 40% (w/w), and poloxamer-407 in an amount of between 5% (w/w) to 15% (w/w).

16. A solid, oral pharmaceutical dosage form according to claim 15, wherein the formulation further comprises one or more pharmaceutically acceptable emulsifier and/or surfactant.

17. A solid, oral pharmaceutical dosage form according to claim 16, wherein the pharmaceutically acceptable emulsifier and/or surfactant is selected from the group consisting of: lecithin, glyceryl monostearate, methylcellulose, sodium lauryl sulfate, sodium oleate, sorbitan monopalmitate, sorbitan monostearate, sorbitan tristearate, tragacanth, triethanolamine oleate, polyethylene sorbitan monolaurate, polyethylene glycol (PEG), macrogol 3350, macrogol 4000, macrogol 6000, detergents, polysorbate 80 (polyoxyethylene sorbitan monooleate), polysorbate 20 (polyoxyethylene sorbitan monolaurate), cetearyl glucoside, polyglucosides, sorbitan monooleate (Span® 80), sorbitan monolaurate (Span® 20), polyoxyethylene monostearate (Myrj 45), polyoxyethylene vegetable oil (Emulphor®), cetyl piridinium chloride, polysaccharides gums, xanthan gums, tragacanth, gum arabica, and acacia.

18. A solid, oral pharmaceutical dosage form according to claim 16, wherein the pharmaceutically acceptable emulsifier and/or surfactant is present in an amount of between 0.1% (w/w) to 35% (w/w) of the total composition.

19. A solid, oral pharmaceutical dosage form according to claim 15, wherein the dosage form is formulated for administration as a dosage form selected from the group consisting of: tablet, pill, pellet, capsule, powder, lozenge, granule, and sustained-release formulation.

20. A solid, oral pharmaceutical dosage form according to claim 15, wherein the dosage form is formulated for administration before food, with food or after food.

21. A solid, oral pharmaceutical dosage form according to claim 15, wherein the cannabidiol is present in an amount of 250 mg per unit dose.

22. A solid, oral pharmaceutical dosage form according to claim 15, wherein the dosage form produces an AUC₀₋ₜ which is between 80% and 125% of the AUC₀₋ₜ of a reference compound Epidiolex® following administration of the dosage form to a subject.

23. A solid, oral pharmaceutical dosage form according to claim 15, wherein the dosage form produces a Cₘₐₓ which is between 80% and 125% of the Cₘₐₓ of a reference compound Epidiolex® following administration of the dosage form to a subject.

24. A method of treatment comprising administering to a subject a therapeutically effective amount of the solid oral dosage form according to claim 15.
